(19) United States
(12) Patent Application Publication
(10) Pub. No.: US 2009/0298065 A1
(43) Pub. Date: Dec. 3, 2009

(54) Methods for Identifying Functional Noncoding Sequences

(75) Inventors: Andrew S. McCallion, Baltimore, MD (US); Shannon Fisher, Baltimore, MD (US); Elizbeth Anne Grice, Pasadena, MD (US)

Correspondence Address: Foley Hoag, LLP (w/JHV), World Trade Center West, 155 Seaport Blvd, Boston, MA, 02210-2600, US

(73) Assignee: The John Hopkins University, Baltimore, MD (US)

(21) Appl. No.: 12/160,053

(22) PCT Filed: Jan. 5, 2007
(86) PCT No.: PCT/US07/60169

§ 371 Date: Feb. 26, 2009

Related U.S. Application Data
(60) Provisional application No. 60/756,290, filed on Jan. 5, 2006.

(51) Int. Cl. C12Q 1/68; C12N 15/63

(52) U.S. Cl. 435/6; 435/320.1

(57) ABSTRACT

The present invention relates to methods for identifying functional noncoding human sequences. Methods may comprise one or more of the following: a comparative genomic sequence analysis step, a genetic analysis step, and a functional analysis step. The functional analysis step comprises transposon-based transgenesis in zebrafish. Also disclosed here in a transposon-based vector to facilitate efficient transgenesis in zebrafish.

### RELATED APPLICATIONS

This application claims the benefit of priority to U.S. Provisional Application 60/756,290, filed Jan. 5, 2006; the contents of which are hereby incorporated by reference in their entirety.

### BACKGROUND OF THE INVENTION

Evolutionary sequence conservation is recognized as a reliable indicator of both coding and noncoding functional sequences. Consistent with this hypothesis, coding sequences may be readily identified based on evolutionary conservation. However, of the five percent of the human genome that is predicted to be functional based on conservation alone, less than one-third actually encodes protein. The remainder, conserved noncoding sequences, are frequently hypothesized to determine tissue specificity, timing, and levels of gene expression (Pennacchio, L. and Rubin, E., (2001) Nat. Rev. Genet. 2:100-9; Waterston, R. et al. (2002) Nature 420:520-62) among other roles. Functionally constrained non-coding are also defined as evolving more slowly than neutral (non-functional) sequences (Kimura, M. and Ota, T. (1971) Nature 229: 467-9).

The identification of putative noncoding regulatory elements has been facilitated by analysis of multiple orthologous genomic sequence intervals and the rapid development and refinement of computational tools. However, the ability to assess and ultimately to predict the biological functions of conserved non-coding sequences remains extremely limited, hampered by inefficient methods for functionally testing computational predictions. Cell culture assays permit analysis of large numbers of sequences, but overlook the complexity of developmental and tissue specific gene regulation. Functional analyses in vivo typically rely on transgenesis in mice, which, although highly informative, is costly and labor intensive, frequently precluding comprehensive analysis of even a single locus. Transgenesis has also been deployed in non-rodent vertebrates, such as zebrafish and Xenopus. However, these approaches are limited by reliance on expression from episomal DNA and visually inaccessible Xenopus embryos. Additionally, standard DNA transgenesis in zebrafish generates highly mosaic G₀ embryos, expressing transgene in <10% of appropriate cells. This high degree of mosaicism has necessitated strategies such as the reconstruction of overall expression patterns from scattered positive cells in numerous Go embryos (Woolfe, A. et al. (2005) PLoS Biol. 3:e7).

To date, only a small fraction of conserved noncoding sequences have been functionally characterized (Oeltjen, J. et al. (1997) Genome Res. 7:315-29; Loots, G. et al. (2000) Science 288:136-40; Pennacchio, L. et al. (2001) 294:169-73; Kellis, M. et al. (2003) Natur 423:241-54; Frazer, K. et al. (2004) Nucleic Acid Res. 32:W273-9). The paucity of functional data for noncoding sequences represents a substantial impediment to evaluating the potential role of noncoding variation in human disease. In fact, despite the recognition that mutations in functional noncoding sequences are predicted to play a significant role in human disease, few have thus far been identified (≦1% of known human mutations). Until now the challenge of examining sufficient numbers of noncoding sequences identified under differing sequence conservation stringencies has appeared insurmountable. Thus, there remains a significant interest in efficiently identifying and characterizing functional noncoding sequences.

### SUMMARY OF THE INVENTION

The present invention provides in part methods for identifying functional noncoding sequences. In one aspect, a method for identifying a functional noncoding DNA sequence comprises one or more of the following steps: identifying a putative functional noncoding interval; cloning the putative functional noncoding interval into a transposon-based vector; expressing the vector in a zebrafish; and monitoring the expression of a reporter in the zebrafish, wherein expression of the reporter indicates that the putative functional noncoding interval is a functional noncoding DNA sequence

In one embodiment, the method comprises a comparative genomic sequence analysis and transposon-based transgenesis in zebrafish to identify functional noncoding sequences.

In certain embodiments, the method comprises identifying a functional noncoding DNA sequence comprising one or more of the following the steps of: identifying a putative functional noncoding interval by comparative sequence analysis; cloning the putative functional noncoding interval into a transposon-based vector; expressing the vector in zebrafish embryos; and monitoring the expression of a reporter in the zebrafish, wherein expression of the reporter indicates that the putative functional noncoding interval is a functional noncoding DNA sequence.

In one embodiment, the comparative sequence analysis comprises comparing orthologous sequences to identify a putative functional noncoding interval. Orthologous sequences are compared to identify conserved regions within noncoding sequences. In some embodiments, putative functional intervals may be classified into one or more of the following categories: coding, noncoding, functional, and non-functional sequences.

In some embodiments, the compared orthologous sequences are vertebrate sequences. In other embodiments, the compared orthologous sequences are mammalian sequences. It other embodiments, the compared orthologous sequences are non-mammalian sequences.

In some embodiments, the putative functional noncoding intervals are vertebrate sequences. In certain embodiments, the putative functional noncoding intervals are mammalian sequences. Mammalian sequences may be human, non-human primates, ovine, bovine, ruminants, caprine, equine, canine, feline, aves, porcine, murine, or marsupial sequences. In other embodiments, the putative functional noncoding interval is from non-mammalian species including, but not limited to teleosts, cartilaginous fish, amphibians, or avians. In one embodiment, the putative functional noncoding interval is from zebrafish.

In another embodiment, the invention provides a method for identifying functional noncoding sequences comprising one or more genetic analyses and transposon-based transgenesis in zebrafish to identify functional noncoding sequences. In certain embodiments, functional noncoding intervals may be identified using one or more genetic analysis, e.g., of transmission disequilibrium tests (TDTs), linkage analyses, or association studies.

In one embodiment, the method comprises identifying a functional noncoding DNA sequence comprising one or more of the following the steps of: identifying a putative functional noncoding interval by one or more genetic tests; cloning the putative functional noncoding interval into a transposon-based vector; expressing the vector in zebrafish embryos; and monitoring the expression of a reporter in the zebrafish, wherein expression of the reporter indicates that the putative functional noncoding interval is a functional noncoding DNA sequence.

In certain embodiments, putative functional noncoding intervals identified by one or more genetic tests may be enriched by comparing orthologous sequences to refine a putative functional interval. In certain embodiments, at least one orthologous sequences is compared to refine the functional noncoding interval. A functional noncoding interval may be refined by at least 50 fold, at least 40 fold, at least 30 fold, at least 20 fold, at least 10 fold, or at least 5 fold.

In other embodiments, putative functional noncoding intervals identified by one or more genetic tests are not enriched by comparative sequence analysis and are evaluated for enhancer activity in a non-biased manner.

In certain embodiments, a sequence may not be analyzed, e.g., to determine whether it is conserved or not across species prior to functional analysis. In certain embodiments, a method comprises introducing a sequence of interest into a vector, e.g., a Tol2 vector and determining whether the sequence is transcriptionally functional.

In some embodiments, functional noncoding intervals are positive regulatory elements, such as enhancers of gene transcription.

Also provided are a transposon-based vectors for expressing putative functional noncoding intervals in zebrafish. In one embodiment, the transposon-based vector is a Tol2 vector. In certain embodiments, the Tol2 vector comprises one or more of a cis-sequence for transposition, a Gateway® ccdB recombination cassette, a mouse cFos minimal promoter, and a reporter gene. In some embodiments, the reporter gene is a fluorescent reporter gene. In one embodiment, the reporter gene is enhanced green fluorescent protein (EGFP).

In one embodiment, the Tol2 vector comprises SEQ ID NO:1 or 2 or a portion thereof. Other vectors may comprise one or more sequences that are at least about 80%, 90%, 95%, 98%, or 99% identical to one or more sequences of SEQ ID NO: 1 or 2. A vector may also comprise or consist of, or consist essentially of, a sequence that is at least about 80%, 90%, 95%, 98%, or 99% identical to SEQ ID NO: 1 or 2.

In another aspect, the invention provides kits for identifying functional noncoding DNA sequences. In one embodiment, a kit may comprise a vector comprising SEQ ID NO:1 and instructions for use. In another embodiment, a kit may comprise a vector comprising SEQ ID NO:2 and instructions for use. In some embodiments, a kit may comprise a vector comprising SEQ ID NO:1 and a vector comprising SEQ ID NO:2. A kit may comprise another reagent, such as an RNA encoding transposase. A kit may still further comprise reagents for cloning putative functional noncoding intervals into the vector and/or reagents for injecting the vector into zebrafish.

Other features and advantages of the invention will be apparent from the following detailed description, and from the claims.

### BRIEF DESCRIPTION OF THE FIGURES

FIG. 1 is a schematic diagram depicting the cloning of a conserved non-coding sequence into a Tol2 transposon expression vector. Conserved non-coding sequences are identified by sequence alignment, in this case using the VISTA server. Primers that contain 5′ attB sequences are designed to amplify the conserved non-coding sequences. The ensuing PCR product is then inserted into an entry vector (pDONR™221) via BP recombination. The resulting construct is recombined with the destination vector (pGW_cfosEGFP) by LR recombination, so that the conserved non-coding sequence is placed in the context of a c-fos minimal promoter driving EGFP expression. After purification and quantification, the construct is ready for injection into zebrafish embryos.

FIG. 2 is a nucleotide sequence for a Tol2 expression vector (SEQ ID NO:1). This sequence provides the Gateway® cassette in the forward orientation.

FIG. 3 is a nucleotide sequence for a Tol2 expression vector (SEQ ID NO:2). This sequence provides the Gateway® cassette in the reverse orientation.

FIG. 4 depicts a comparative sequence analysis of teleost ret loci revealing putatively functional noncoding sequences. VISTA plot displaying the alignment of the zebrafish ret locus with the orthologous fugu region. Red peaks represent conserved noncoding sequences; shaded green boxes represent zebrafish conserved sequence (ZCS) amplicons. Boxes bordered by dashed lines denote amplicons containing ≧2 conserved sequences. ret exons are denoted by blue peaks. Red peaks boxed and shaded in blue denote 5′ and 3′ flanking genes pcbd and galnact2, respectively.

FIG. 5 shows that conserved noncoding sequences at the zebrafish and human ret loci drive reporter expression in zebrafish embryos consistent with the endogenous gene. Shown are GFP expression patterns in representative G₀ embryos. (A to D) Zebrafish elements drive expression in: (A) bilateral olfactory pits (arrowheads; ZCS-83); (B) hindbrain neuron consistent with nVII facial motor neuron (arrowhead; ZCS-19.7); (C) pronephric duct before 24 hours. (arrowhead; ZCS-34); (D) pronephric duct at 3 days; (arrowheads; ZCS-7.6). Human elements drive expression in (E), pituitary (encircled, HCS+16); (F) dorsal spinal cord neurons (arrowheads, HCS-32; fp, floor plate; nc, notochord); (G) pronephric duct (arrowheads) and enteric neurons (open arrowhead; HCS+9.7); (H) enteric neurons (open arrowheads, HCS+9.7).

FIG. 6 shows mosaic G₀ expression accurately reflects expression in G₁ fish. (A) ZCS-35.5 G₀ embryos display GFP in cells of the anterior (open arrowhead) and posterior (solid white arrowhead) lateral line placode ganglia. (B) ZCS-35.5 G₁ embryos display GFP in the anterior (open arrowhead) and posterior (solid white arrowhead) lateral line placode ganglia, as in (A). (C) GFP detected by in situ hybridization (ISH) in the distal pronephric duct of ZCS+7.6 G₁ embryo at 24 hours, consistent with ret expression at the same stage (D). (E and F) GFP detected by ISH in the pituitary (open arrowhead), trigeminal nuclei (arrow), and migrating nVII facial motor neurons [arrowhead in (E, F)] of a HCS+16 G₁ embryo. (G) GFP detected by ISH in the retina of G₁ ZCS-19.7 embryo.

FIG. 7 is a series of photographs showing examples of tissue-specific regulatory control provided by conserved non-coding sequences amplified from Human (human conserved sequence; HCS), mouse (mouse conserved sequence; MCS) and Zebrafish (zebrafish conserved sequence; ZCS) genomes. (A) Reporter expression in cranial ganglia (CG) driven by a zebrafish conserved non-coding sequences amplified from sequence flanking the ret proto-oncogene. (B) Reporter expression throughout the hindbrain (Rhombomeres 1-7) and spinal column driven by a zebrafish conserved non-coding sequences amplified from sequence flanking the phox2b transcription factor. (C) Anterior spinal column (ASC) expression similarly driven by another phox2b conserved non-coding sequence. (D) Myelinating oligodendrocytes (Olig) and Schwann cells (Sch) identified using a conserved non-coding sequence amplified from the mouse Sox10 transcription factor gene. (E) Signal in enteric nervous system (ENS) neuronal precursors generated using a conserved non-coding sequence amplified from the zebrafish phox2b transcription factor gene. (F-G) Dopaminergic populations of the ventral diencephalon (VeDi) identified using conserved non-coding sequences amplified from the zebrafish phox2b (F) and human NR4A2 (G) genes; also identified are hindbrain (Hb; F) and Olfactory (Olf; G) neuronal populations. (H) Reporter expression driven by a human conserved non-coding OSX enhancer sequence in forming bone. (I) Pan-neural crest reporter expression driven by a mouse conserved non-coding sequence at Sox10 (arrowheads, migratory chains of crest; arrows, pre-migratory crest). (J) Hind brain and spinal reporter expression driven by a human conserved non-coding sequence amplified from the interval around PHOX2B.

### DETAILED DESCRIPTION OF THE INVENTION

### 1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, the term “genome” is intended to mean the full complement of chromosomal DNA found within the nucleus of a eukaryotic cell. The term can also be used to refer to the entire genetic complement of a prokaryote, virus, mitochondrion or chloroplast or to the haploid nuclear genetic complement of a eukaryotic species.

As used herein, the term “genomic DNA” or “gDNA” is intended to mean one or more chromosomal polymeric deoxyribonucleotide molecules occurring naturally in the nucleus of a eukaryotic cell or in a prokaryote, virus, mitochondrion or chloroplast and containing sequences that are naturally transcribed into RNA as well as sequences that are not naturally transcribed into RNA by the cell. A gDNA of a eukaryotic cell contains at least one centromere, two telomeres, one origin of replication, and one sequence that is not transcribed into RNA by the eukaryotic cell including, for example, an intron or transcription promoter. A gDNA of a prokaryotic cell contains at least one origin of replication and one sequence that is not transcribed into RNA by the prokaryotic cell including, for example, a transcription promoter. A eukaryotic genomic DNA can be distinguished from prokaryotic, viral or organellar genomic DNA, for example, according to the presence of introns in eukaryotic genomic DNA and absence of introns in the gDNA of the others.

As used herein, “a putative functional interval,” such as a “putative functional noncoding interval” refers to any sequence interval that has functional activity, e.g., an enhancer for gene transcription. In one embodiment, putative functional intervals may be identified by comparative sequence analysis to identify conserved sequence regions. In another embodiment, putative functional intervals may be identified by genetic analyses, including, for example, transmission disequilibrium tests (TDTs), linkage, or association studies. These methods are useful in predicting functional intervals. Sequencing putative functional intervals to identify mutations within the interval can be by any known or future developed sequencing methods.

“Mutation,” as used herein, refers, for example, to a polymorphism or marker that occurs in those at risk of developing a disease, is associated with a disease, and contributes to disease risk or causative of a disease. In certain instances, the mutation may be strongly correlated with the presence of a particular disorder (e.g., the presence of such mutation indicating a high risk of the subject being afflicted with a disease). However, “mutation” as used herein can also refer to a specific site and type of polymorphism or marker, without reference to the degree of risk that particular mutation poses to an individual for a particular disease. Mutations, as used herein, are over-represented in affected subjects as compared to normal subjects and may be associated with a multigenic disease. The multigenic disease may comprise, for example, one or more of mental illness, cancer, cardiovascular disease, congenital anomalies, metabolic disorder include but not limited to diabetes, susceptibility to infection, drug response, or drug tolerance. Mutations may be one or more of associated with a disease susceptibility, causative of disease, or contributory to disease and the like. Mutations, as used herein may comprise a single nucleotide polymorphism, a multi-nucleotide polymorphism, an insertion, a deletion, a repeat expansion, genomic rearrangements, or segmental amplification.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The term “upstream” is used herein to refer to a location which, is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995). The terms “complementary” or “complement thereof are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

A “promoter” refers to a DNA sequence recognized by the synthetic machinery of the cell required to initiate the specific transcription of a gene.

A sequence which is “operably linked” to a regulatory sequence such as a promoter means that said regulatory element is in the correct location and orientation in relation to the nucleic acid to control RNA polymerase initiation and expression of the nucleic acid of interest. As used herein, the term “operably linked” refers to a linkage of polynucleotide elements in a functional relationship. For instance, a promoter or enhancer is operably linked to a coding sequence if it affects the transcription of the coding sequence. More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide. The TDT (Spielman et al. (1993) Am J Hum Genet 52: 506-16) is a test for both association and for linkage, more specifically, it tests for linkage in the presence of association. Thus, if association does not exist at the locus of interest, linkage will not be detected even if it exists. It is for this reason that the test has been included in this section. It may be used as an initial test, but is more commonly used when tentative evidence for association has already been identified. In this case, a positive result will not only confirm the initial association, but also provide evidence for linkage.

As used herein, the term “detecting” is intended to mean any method of determining the presence of a particular molecule such as a nucleic acid having a specific nucleotide sequence. Techniques used to detect a nucleic acid include, for example, hybridization to the sequence to be detected. However, particular embodiments of this invention need not require hybridization directly to the sequence to be detected, but rather the hybridization can occur near the sequence to be detected, or adjacent to the sequence to be detected. Use of the term “near” is meant to imply within about 150 bases from the sequence to be detected. Other distances along a nucleic acid that are within about 150 bases and therefore near include, for example, about 100, 50 40, 30, 20, 19, 18, 17, 16, 15, 14, 13, 12, 11, 10, 9, 8, 7, 6, 5, 4, 3, 2, or 1 bases from the sequence to be detected. Hybridization can occur at sequences that are further distances from a locus or sequence to be detected including, for example, a distance of about 250 bases, 500 bases, 1 kilobase or more up to and including the length of the target nucleic acids or genome fragments being detected.

Examples of reagents which are useful for detection include, but are not limited to, radiolabeled probes, fluorophore-labeled probes, quantum dot-labeled probes, chromophore-labeled probes, enzyme-labeled probes, affinity ligand-labeled probes, electromagnetic spin labeled probes, heavy atom labeled probes, probes labeled with nanoparticle light scattering labels or other nanoparticles or spherical shells, and probes labeled with any other signal generating label known to those of skill in the art. Non-limiting examples of label moieties useful for detection in the invention include, without limitation, suitable enzymes such as horseradish peroxidase, alkaline phosphatase, beta-galactosidase, or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the [omicron].sup.th Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S ³H.

### 2. Methods of Identifying Functional Noncoding Sequences

The ability to rapidly examine the regulatory potential of all putative functional noncoding sequences in a cost-effective manner is essential for a full understanding of their biological role and to further refine the computational tools used in their prediction. Described herein is an approach, using a high-efficiency vector in visually accessible zebrafish embryos, which will facilitate large-scale functional analysis of sequences from vertebrate genomes. The assay is designed to identify positive regulatory elements, e.g. enhancers of gene transcription.

In certain embodiments, negative regulatory sequences may also be readily evaluated in a targeted tissue-specific manner. For example, tissue-specific repression may be evaluated by combining an enhancer sequence with known expression that includes and extends beyond a tissue of interest, e.g., heart and eye. These sequences may be cloned with other known enhancer sequences to look for repression in the heart. Continued expression (i.e., signal) in the eye would indicate success and serve as an assay control, while repression in the heart would indentify the desired biological activity.

The use of this technology may yield new in vivo substrates for lineage analysis during development and disease processes; may facilitate the elucidation of complex regulatory networks; and may be used to support ongoing activities to permit functional annotation of vertebrate genomes.

One aspect of the invention is to address the issue of extreme G₀ mosaicism in the visually accessible zebrafish embryo. As described herein, a reporter vector was developed to functionally examine putative enhancers in transgenic zebrafish. This vector was based on the Tol2 transposon, originally identified from the medaka Orzyas latipes (Koga, A. et al. Nature 383, 30 (1996)). Previously described methods that were developed to increase the efficiency of zebrafish transgenesis were based on the Sleeping Beauty transposon (Davidson, A. et al. Dev Biol 263, 191-202 (2003); Ivics, Z. et al. Cell 91, 501-10 (1997)) or relied on I-SceI meganuclease digestion of injected DNA (Thermes, V. et al. Mech Dev 118, 91-8 (2002)). However, the reported rates of germline transmission for Tol2 vectors are higher (Kawakami, K. et al. Dev Cell 7, 133-44 (2004)) than those rates reported for these alternative methods. In addition, substantially greater expression of a ubiquitous control construct was observed in G₀ embryos with a Tol2 vector than with one based on Sleeping Beauty.

As described herein, a smaller Tol2 vector was constructed. The Tol2 vector comprises an essential cis-sequences for transposition in addition to a Gateway® ccdB recombination cassette and mouse cFos minimal promoter (Dorsky, R. et al. (2002) Dev. Biol. 241:229-37) placed upstream of the EGFP gene. Without the addition of further sequences, the cFos minimal promoter fails to drive reporter gene expression in transgenic zebrafish. Inserting a regulatory element with positive activity, e.g. an enhancer sequence, into the Gateway® cassette results in EGFP expression reflecting the normal regulatory activity of the enhancer, while insertion of a sequence with negative or no regulatory activity will not lead to detectable EGFP.

A Tol2 vector may comprise SEQ ID NO:1 or SEQ ID NO:2. The vector comprising SEQ ID NO:1 comprises the Gateway® cassette in the forward orientation. The vector comprising SEQ ID NO:2 comprises the Gateway® cassette in the reverse orientation. For SEQ ID NOs:1 and 2, base pairs 2208-2791 correspond to Tol2 transposon sequences from left arm; base pairs 2794-4504 correspond to the Gateway cassette (either in forward (SEQ ID NO:1) or reverse (SEQ ID NO:2) orientation); base pairs 4508-4605 correspond to the cFos minimal promoter; base pairs 4612-5625 correspond to EGFP coding sequence and polyadenylation sequence; and base pairs 5632-6139 correspond to Tol2 transposon sequences from right arm. The remainder of the sequence (1-2207 and 6140-6797) is the backbone vector, pBluescript KS+.

One of skill in the art will readily understand that the Tol2 vectors described herein may be modified in a number of ways. Modifications may include individual nucleotide substitutions to a Tol2 vector or insertions or deletions of one or more nucleotides in the vector sequences. Modifications to a Tol2 vector sequence that alter (i.e., increase or decrease) expression of a sequence interval (e.g., alternative promoters), provide greater cloning flexibility (e.g., alternative multiple cloning sites), provide greater experimental efficiency (e.g., alternative reporter genes), and/or increase vector stability are contemplated herein.

In one embodiment, a Tol2 vector of the invention may be modified to replace the Gateway cassette with a multi-cloning sequence, containing restriction enzyme sites for insertion of potential enhancers through standard ligation. For example, base pairs 2794-4504 corresponding to the Gateway cassette (either in forward (SEQ ID NO:1) or reverse (SEQ ID NO:2) orientation) may be replaced with any multi-cloning site that may be used to insert putative functional noncoding intervals.

In another embodiment, a Tol2 vector of the invention may be modified to eliminate the cFos minimal promoter sequence, to allow testing of an enhancer-promoter combination including the endogenous gene promoter. For example, base pairs 4508-4605 corresponding to the cFos minimal promoter may be replaced with an alternative promoter sequence.

In another embodiment, a Tol2 vector of the invention may be modified to use alternative minimal promoters, including those derived from the mouse Hsp68 gene and the zebrafish hsp70 genes.

In another embodiment, a Tol2 vector of the invention may be modified to use alternative reporter genes, including genes encoding other fluorescent proteins such as mCherry, or enzymes such as β-gal and alkaline phosphatase. In certain embodiments, fluorescent reporters may replaced with alternate fluorescent reporters with shorter or longer protein half-life allowing more precise evaluation of the timing of regulatory control and tracking cell migration and lineage, respectively. A reporter may be also be replaced by cassettes encoding protein substrates which allow observation (direct or indirect) of response based on cell/biochemical activity, e.g., driving such a reporter in noradrenergic populations would allow analysis of which sub-populations were responding appropriately to chemical stimuli e.g. in screens of chemical libraries to identify potential therapeutic chemical targets/leads.

Further, a Tol2 vector of the invention may be modified to create a “driver” construct encoding Gal4 or a variant such as a Gal4-VP16 fusion protein instead of EGFP. A transgenic line made with such a driver could then be crossed to any number of responder lines carrying genes under control of the UAS enhancer element, resulting in tissue-specific expression of the responder transgene driven by Gal4.

In certain embodiments, a Tol2 vector of the invention may be modified to in one or more ways, e.g., a Tol2 vector may be modified to use both an alternative minimal promoter and an alternative reporter gene or a Tol2 vector may be modified to replace the Gateway cassette with a multi-cloning sequence and include an alternative minimal promoter and/or an alternative reporter gene. In still further embodiments, a Tol2 vector may be modified to replace the Gateway cassette with a multi-cloning sequence and to include an alternative minimal promoter and/or an alternative reporter gene and/or driver construct encoding Gal4 or a variant such as a Gal4-VP16 fusion protein instead of EGFP.

Modifications to a Tol2 vector of the invention may result in a vector that is at least 50% identical, at least 60% identical, at least 70% identical, at least 80% identical, at least 90% identical, at least 95% identical, at least 96% identical, at least 97% identical, at least 98% identical, or at least 99% identical to SEQ ID NO:1 or SEQ ID NO:2 or a portion thereof.

Also described herein are methods of identifying functional noncoding regulatory sequences in vertebrates. The methods may employ a combination of human genetic, comparative genomic, functional, and/or population genetic analyses. In one embodiment, the method comprises identifying a functional noncoding DNA sequence comprising one or more of the steps of: identifying a putative functional noncoding interval; cloning the putative functional noncoding interva into a transposon-based vector; expressing the vector in zebrafish embryos; and monitoring the expression of a reporter in the zebrafish, wherein the expression of the reporter indicates that the putative functional noncoding interval is a functional noncoding DNA sequence.

In one embodiment, the comparative genomic sequence and a functional analysis can be used to identify functional noncoding sequence intervals. In another embodiment, one or more genetic analysis and a functional analysis can be used to identify functional noncoding intervals.

The methods described herein may comprise classifying sequence intervals into one or more of the following: coding, noncoding, functional, and non-functional sequences. Functional noncoding regulatory sequences may include positive regulatory elements and negative regulatory elements. Functional noncoding sequences are referred to herein as “functional noncoding intervals.” Functional noncoding intervals may be bound between coding regions, a coding region and an adjacent noncoding sequence, or adjacent noncoding sequences flanking both sides of the functional noncoding interval.

In certain embodiments, comparative sequence analysis may be used to identify and/or refine putative functional noncoding intervals. In general, conserved noncoding sequences can be identified using multiple sequence alignment programs known in the art. For example, functional noncoding intervals may be identified by comparing orthologous sequences from multiple organisms to identify and/or refine a putative functional interval. Sequences encompassing the putative functional noncoding intervals may be identified and/or refined by creating a multiple sequence alignment.

Multiple sequence alignments may be readily performed using the publicly available UCSC genome browser (available on the world wide web with the extension genome.uscs.edu), which permits a person skilled in the art to align and evaluate sequences in silico with sophisticated tools such as phastCons (Siepel, A. et al. Genome Res 15, 1034-50 (2005)). In addition, there are numerous freely available stand-alone alignment algorithms that may be used to predict functional sequences predicated on overlapping but subtly different parameters. Some of the more commonly used algorithms include VISTA (Frazer, K. et al. Nucleic Acids Res 32, W273-9 (2004)), MultiPipmaker (Schwartz, S. et al. Genome Res 10, 577-86 (2000)), Multi-species Conserved Sequences (Margulies, E. et al. Genome Res 13, 2507-18 (2003)), Regulatory Potential (Kolbe, D. et al. Genome Res 14, 700-7 (2004)) and LAGAN (Brudno, M. et al. BMC Bioinformatics 4, 66 (2003)).

Functional noncoding intervals may be identified in any vertebrates. Vertebrate sequences comprise mammalian, reptilian, avian, amphibians, or osteichthyes. Mammalian sequences may include human sequences and non-human sequences. Non-human sequences include rodents, non-human primates, ovines, bovines, ruminants, lagomorphs, porcines, caprines, equines, canines, felines, aves, piscines, marsupials, etc. Exemplary non-human mammals are porcines (e.g., pigs), murines (e.g., rats, mice, and lagomorphs (e.g., rabbits)), and non-human primates (e.g. monkeys and apes). Nonmammlian sequences may include teleosts, cartilaginous fish, amphibians, or avians. Exemplary lower vertebrates sequences include zebrafish (a teleost) sequences.

Orthologous sequence comparison may comprise a comparison of any or all vertebrate sequences. For example, orthologous sequence intervals may be identified following a comparison of all known sequences for a specified gene locus, all vertebrate and/or mammalian sequences for a specified gene locus, or subset of all vertebrate and/or mammalian sequences for a specified gene locus.

Orthologous sequence comparisons may also be based on single celled organisms, e.g., yeast, bacteria, viruses, and the like.

It will be understood that the invention provides systems that may be employed to compare the orthologous sequences. The systems may be machines as well as software tools and can include devices for processing sequence data as well as data visualization tools which can highlight patterns in data that is visually displayed. The system may comprise a conventional data processing platform such as an IBM PC-compatible computer running the Windows operating systems, or a SUN workstation running a Unix operating system. Alternatively, the system can comprise a dedicated processing system that includes an embedded programmable data processing system. For example, the system can comprise a single board computer system that has been integrated into a system for sequencing genomic data, identifying SNPs or markers, collecting expression data, or for performing other laboratory processes. The system may also be able to process classifying the sequence data into one or more of coding, non-coding, functional and non-functional sequences.

Also provided are methods for identifying functional noncoding sequences comprising one or more genetic analyses and transposon-based transgenesis in zebrafish. In certain embodiments, functional noncoding intervals may be identified using one or more genetic tests, e.g., of transmission disequilibrium tests (TDTs), linkage, or association studies.

Multi-allele Transmission Disequilibrium Test (TDT). TDT is at widely used method for family-based genetic study (Spielman et al., Transmission test for linkage disequilibrium: the insulin gene region and insulin-dependent diabetes mellitus (IDDM), Am. J. Hum. Genet, 1993 March; 52 (3):506-16), where parents and children in a family are typed. Testing for linkage in the presence of linkage disequilibrium (association), TDT can be very powerful to identify susceptibility locus, especially when the effect is small, as is often the case with complex genetic trait. Although the original TDT test was developed to analyze biallelic markers, new statistics have been developed to accommodate the availability of multiallelic markers or haplotypes (Spielman et al., The TDT and other family-based tests for linkage disequilibrium and association, Am. J. Hum. Gent., 1996 November; 59 (5):983-9; Curtis and Sham, Model-free linkage analysis using likelihoods, Am. J. Hum. Genet., 1995 September; 57(3):703-16; Bickeboller et al., Statistical properties of the allelic and genotypic transmission/disequilibrium test for multiallelic markers, Genet. Epidemiol., 1995; 12(6):865-70). Based on survey performed by Kaplan (Kaplan et al., Power studies for the transmission/disequilibrium tests with multiple alleles, Am. J. Hum. Genet., 1997 March; 60(3):691-702) on those methods, we have chosen the marginal statistics with only heterozygous parents (T.sub.mhet) by Spielman and Ewens (Spielman et al., The TDT and other family-based tests for linkage disequilibrium and association, Am. J. Hum. Genet., 1996 November; 59(5):983-9), because it has equivalent power to the other multi-allelic tests and gives a valid chi-square test of linkage. Multi-allele TDT can be readily applied to patterns because of the multi-allele or multi-genotype nature of a pattern. In a TDT test on a pattern, each observed permutation of a pattern is treated as column and row headings in a TDT contingency table. Corresponding chi-square value is calculated based on described (Spielman et al., The TDT and other family-based tests for linkage disequilibrum and association, Am. J. Hum. Genet., 1996 November; 59 (5):983-9) and P value is assigned according to default or reference distribution simulated by Monte Carlo. This statistics can only be applied to patterns identified in a family-based association study design.

The Quantitative Transmission Disequilibrium Test (OTDT) Analysis was proposed by George et al. [1999] was used to conduct QTDT analysis. This test detects linkage in the presence of association. This test detects linkage in the presence of association. The maximum likelihood estimates of the parameters and the standard errors of the estimates are computed by numerical methods. These procedures are implemented in the program ASSOC of the S.A.G.E. [1998] software package. Single permutation tests have been used in mapping studies before (Churchill and Doerge 1994, Laitinen et al. 1997, Long and Langley 1999). However, if more complex data is to be analyzed, these single permutation tests are too expensive and computationally very ineffective and even inoperative.

The Haplotype-based Haplotype Relative Risk (HHRR) test is another method for family-based studies (Terwilliger et al., A haplotype-based “haplotype relative risk” approach to detecting allelic associations, Hum. Hered., 1992; 42(6):337-46, 1992). It is a variation of the Haplotype Relative Risk (HRR) method, which is genotype-based. In Rubinstein's Genotype-based haplotype relative risk (GHRR) method, the affected children's genotypes at a marker locus are used as cases and artificial genotypes made up of the alleles not transmitted to the children from their parents are used as controls. For each haplotype of interest, a 2×2 contingency table is constructed and used to record the number of cases and controls with or without that haplotype. In contrast, HHRR utilizes haplotypes rather than genotypes. In particular, transmitted chromosomes are treated as cases and untransmitted chromosomes are used as controls, A 2×2 table is constructed the same as for GHRR. HHRR can be extended to be applied to patterns because of the similarity between a pattern and a multi-marker haplotype. In a HHRR test for a pattern, the observed counts for the pattern in cases and in controls and the observed counts for all other permutations on markers in that pattern in cases and controls are recorded in the 2×2 contingency table. Upon the calculation of chi-square values, P values are assigned according to default distribution or reference distribution simulated by Monte Carlo. Statistical significant based on uncorrelated pattern formation (Califano et al., Analysis of gene expression microarrays for phenotype classification, Proc. Int. Conf Intell. Syst. MoI. Biol., 2000; 8:75-85).

“Linked,” as used herein, refers, for example, to a region of a chromosome shared more frequently in family members affected by a particular disease than would be expected by chance, thereby indicating that the gene or genes within the linked chromosome region contain or are associated with a marker or polymorphism that is correlated to the presence of, or risk of, disease. Once linkage is established, for example, by association studies (linkage disequilibrium) can be used to narrow the region of interest or to identify the risk-conferring gene associated with a disease.

“Associated with” when used to refer for example to a marker or polymorphism and a particular gene means that the polymorphism or marker is either within the indicated gene, or in a different physically adjacent gene on that chromosome. In general, such a physically adjacent gene is on the same chromosome and within 2, 3, 5, 10 or 15 centimorgans of the named gene (i.e., within about 1 or 2 million base pairs of the named gene). The adjacent gene may span over 5, 10 or even 15 megabases. Polymorphisms may be functional polymorphisms. “Associated with,” in reference to a mutation being associated with a disease, refers to, for example, a statistical association. A “centimorgan”0 as used herein refers to a unit of measure of recombination frequency. One centimorgan is equal to a 1% chance that a marker at one genetic locus will be separated from a marker at a second locus due to crossing over in a single generation. In humans, one centimorgan is equivalent, on average, to one million base pairs. Markers and polymorphisms of this invention (e.g., genetic markers such as single nucleotide polymorphisms, restriction fragment length polymorphisms and simple sequence length polymorphisms) can be detected directly or indirectly. A marker can, for example, be detected indirectly by detecting or screening for another marker that is tightly linked (e.g., is located within 2 or 3 centimorgans) of that marker. Additionally, the adjacent gene can be found within an approximately 15 cM linkage region surrounding the chromosome, thus spanning over 5, 10 or even 15 megabases.

The presence of a marker or polymorphism associated with a gene linked to, for example, a disease, for example Hirschsprung disease, indicates that the subject is afflicted with the disease or is at risk of developing the disease and/or is at risk of developing the disease. A subject who is “at increased risk of developing a disease” is one who is predisposed to the disease, has genetic susceptibility for the disease and/or is more likely to develop the disease than subjects in which the detected polymorphism is absent. A subject who is “at increased risk of developing a disease at an early age” is one who is predisposed to the disease, has genetic susceptibility for the disease and/or is more likely to develop the disease at an age that is earlier than the age of onset in subjects in which the detected polymorphism is absent. Thus, the marker or polymorphism can also indicate “age of onset” of a disease.

The methods described herein can be employed to screen for any type of disease, including, for example, multigenic diseases, mental illness, cancer, cardiovascular disease, congenital anomalies, metabolic disorder inc but not limited to diabetes, susceptibility to infection, drug response, or drug tolerance, and the like.

As used herein, “predicting a genetic interval for a disease,” refers to, for example, identifying an interval associated with a disease using for example, one or more genetic tests, e.g., of transmission disequilibrium tests (TDTs), linkage, or association studies.

Methods of predicting an interval comprise, for example, multi-analytical approaches including both parametric lod score and non-parametric affected relative pair methods. Maximized parametric lod scores (MLOD) for each marker may be calculated, for example, by using VITESSE and HOMOG program packages (O'Connell & Weeks, Nat. Genet. 11:402 (1995); Ott, Analysis of Human Genetic Linkage. (The Johns Hopkins University Press, Baltimore, Ed. 3, 1999); The MLOD is the lod score maximized over the two genetic models tested, allowing for genetic heterogeneity. Dominant and recessive low-penetrance (affecteds-only) models may be considered. Methods may be further based on prevalence estimates and for example, age-dependent or incomplete penetrance. Disease allele frequencies of 0.001 for the dominant model and 0.20 for the recessive model may be used. Marker allele frequencies may be generated, for example, from related or unrelated individuals. Multipoint non-parametric lod scores (LOD*) may be calculated, for example, using GENEHUNTER-PLUS software (Kong & Cox, Am. J. Hum. Genet. 61:1179 (1997)) and sex-averaged intermarker distances. In contrast to non-parametric linkage approaches which consider allele sharing in pairs of affected siblings [Risch, Am. J. Hum. Genet. 46:222 (1990)], GENEHUNTER-PLUS considers allele sharing across pairs of affected relatives (or all affected relatives in a family) in moderately sized pedigrees.

In one embodiment, the method comprises identifying a functional noncoding DNA sequence comprising one or more of the following the steps of: identifying a putative functional noncoding interval by one or more genetic tests; cloning the putative functional noncoding interval into a transposon-based vector; expressing the vector in zebrafish embryos; and monitoring the expression of a reporter in the zebrafish, wherein expression of the reporter indicates that the putative functional noncoding interval is a functional noncoding DNA sequence.

In certain embodiments, putative functional noncoding intervals identified by one or more genetic tests may be enriched by comparing orthologous sequences to refine a putative functional noncoding interval. In another embodiment, the further refinement of sequence intervals is achieved by further sequence analysis and/or population genetic analysis. In other embodiments, putative functional noncoding intervals identified by one or more genetic tests are not enriched by comparative sequence analysis and are evaluated for enhancer activity in a non-biased manner.

As used herein, “comparing orthologous sequences to refine a putative functional interval,” refers to, for example the use of at least one orthologous sequence to the interval. The orthologous sequence refines the interval, by, for example, revealing the evolutionarily conserved regions of the interval that are more likely to be under selective pressure. Thus, differences or mutations found in these regions are more likely to be associated with disease. One or more orthologous sequences may be compared to the interval for further refining. The comparing can be done by software, hardware or by an individual.

In one embodiment, one orthologous sequence is compared to refine the interval. In another embodiment, at least two orthologous sequences are compared to refine the interval. In one embodiment, the interval is refined by the comparison to one or more orthologous sequences by at least about 50 fold, at least about 40 fold, at least about 30 fold, at least about 25 fold, at least about 20 fold, at least about 15 fold, by at least about 10 fold, or at least about 5 fold.

“Classifying the refined interval,” as used herein refers to, for example, defining function or type of sequence that makes up the interval. The classifications, as indicated above, include, one or more of coding, noncoding, functional and non-functional sequences. For example, noncoding sequences may be classified as functional or non-functional sequences.

In certain embodiments, a sequence interval may be identified or generated by tiling a path of amplicons across an interval. For example, tiling of PCR products may be used to generate a putative functional sequence interval.

In certain embodiments, a sequence interval may not be analyzed, e.g., to determine whether it is conserved or not across species prior to functional analysis. In certain embodiments, a method comprises introducing a sequence interval of interest into a vector, e.g., a Tol2 vector and determining whether the sequence is transcriptionally functional.

The sequence interval of interest may comprise about 0. 1 to 6 kb of DNA. In some embodiments, the sequence interval of interest may comprise about 0. 1 to 5 kb of DNA, about 0.1 to 4 kb of DNA, about 0.1 to 3 kb of DNA, about 0.1 to 2 kb of DNA, about 0.1 to 5 kb of DNA. In other embodiments, the sequence interval of interest may comprise about 1 to5 kb of DNA, about 1 to 4 kb of DNA, about 1 to 3 kb of DNA or about 1 to 2 kb of DNA. In still other embodiments, the sequence interval of interest may comprise about 2 to 5 kb of DNA, about 3 to 5 kb of DNA, or about 4 to 5 kb of DNA.

Also considered herein is the function of multiple human sequences as specific enhancer elements in zebrafish embryos in the absence of detectable sequence conservation across the same evolutionary span. Thus, the utility the method described herein can extend to mammalian loci where the corresponding zebrafish gene has not been characterized, or where sequence conservation is not detected beyond coding exons.

Functional intervals may be further investigated to identify disease intervals in which specific mutations can be identified and characterized. In one embodiment, a method of identifying a mutation in DNA comprises predicting a genetic interval for a disease; comparing orthologous sequences to refine a putative functional interval; and sequencing the putative functional interval in subjects to identify mutations.

In another embodiment, a method of identifying a mutation in DNA, comprises predicting a genetic interval harboring mutations that contribute to disease susceptibility; comparing orthologous sequences to refine a putative functional interval; and sequencing the putative functional interval subjects to identify mutations.

In one embodiment, the predicting comprises one or more of transmission disequilibrium tests (TDTs), linkage, or association studies. In another embodiment, the subjects comprise individuals from affected families. In one embodiment, the subjects comprise affected and unaffected individuals. In another embodiment, mutations are over-represented in affected subjects as compared to normal subjects. In some embodiments, the mutation may be associated with a multigenic disease. In certain embodiments, the multigenic disease may comprise one or more of mental illness, cancer, cardiovascular disease, congenital anomalies, metabolic disorder inc but not limited to diabetes, susceptibility to infection, drug response, or drug tolerance. In another embodiment, the mutations are one or more of associated with a disease susceptibility, are causative of disease, and are contributory to disease.

In one embodiment, the mutation comprises a single nucleotide polymorphism, a multi-nucleotide polymorphism, an insertion, a deletion, a repeat expansion, genomic rearrangements, or segmental amplification.

In certain embodiments, the methods described herein may be used to evaluate the biological and/or pathological impact of variation within a sequence interval. For example, the methods may be used to evaluate a “wild type” sequence identified based on sequence conservation or by other methods and demonstrate that the “wild type” sequence interval has regulatory control. This sequence interval can be obtained in a biological sample from patients and sequenced. Sequence variation can be determined by comparison to the “wild type” sequence interval and frequency of the sequence variation can be measured in patients. Elevated sequence variation may be found in individuals suffering from a disease. Using the methods described herein, the biological activity of the “disease associated” sequence can be determined.

In another embodiment, the methods described herein may be used to evaluate the biological and/or pathological impact of sequence variation within other genic or non-genic sequence in the genome. For example, the methods described herein may be used to evaluate the biological impact of mutations in functional sequences of other disease associated genes.

In another embodiment, the methods described herein may be used to evaluate the biological and/or pathological impact of environmental exposure, such as to toxins, drugs, chemicals, temperature, stress, etc.

In another embodiment, the methods described herein may be used to identify sequence intervals for use in other systems. For example, the methods described herein may be used to identify sequences with cell type specific regulatory control that may be used in in vitro to identify or isolate cells in differentiating mixed populations of cells (e.g., primary, immortalized, stem (human or non-human, such as mouse, embyronic and adult) cells for further analysis, the generation of in vitro phenotypes for drug screening, and/or engraftment analyses (e.g., analyses that may be used to determine therapeutic value, efficacy, and/or safety).

The methods described herein may also comprise the step of amplifying the nucleic acid sequence interval before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197). Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like. PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In yet another embodiment, any of a variety of sequencing reactions known in the art can be used to directly sequence the functional sequence intervals. Exemplary sequencing reactions include those based on techniques developed by Maxim and Gilbert ((1977) Proc. Natl Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO94/16101; Cohen et al. (1996) Adv Chromatogr 36:127-162; and Griffin et al. (1993) Appl Biochem Biotechnol 38: 147-159).

It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out. Single molecule sequencing methods may also be used.

### 3. Evaluation of Putative Functional Noncoding Intervals Using Tol2 Transposon-Mediated Transgenesis in Zebrafish

The method described herein further comprises a functional analysis of the identified sequence interval. In one embodiment, the functional analysis is a transposon-based transgenesis in zebrafish. This approach provides for the rapid examination of the ability of the putative functional noncoding intervals to direct tissue-specific GFP expression in live zebrafish.

Alternative reporters may be used in the described methods. Alternative reporters include enhanced green fluorescent protein (EGFP) variants, such as enhanced red fluorescent protein (ERFP), enhanced yellow fluorescent protein (EYFP), and enhanced blue fluorescent protein (EBFP). Fluorescent reporters may be replaced by fluorescent reporters with shorter or longer protein half-life allowing more precise evaluation of the timing of regulatory control and tracking cell migration, respectively.

Putative functional noncoding intervals (as well as all other sequence intervals that may be identified using the methods described above) are introduced into a Tol2 vector as described above. Following the introduction of putative functional noncoding intervals into the Tol2 vector, the method described herein may be used to create zebrafish transgenics more efficiently.

Exemplary methods for cloning sequence intervals, e.g., putative functional noncoding intervals, into the Tol2 vector and introducing the vector into zebrafish are described below.

### Primer Design for PCR Cloning

Primers are designed to amplify the DNA sequence of interest (e.g., the functional noncoding interval), typically including ≧30 bp flanking DNA on either side of the conserved sequence, since the boundaries of functional elements may not be readily predicted. Clusters of non-coding conserved sequences can be amplified in a single PCR product and their individual roles dissected subsequently if necessary. For primer design, Primer3 (available on the world wide web with the extension frodo.wi.mit.edu/cgi-bin/primer3/primer3_www.cgi) or similar primer design software may be used. To enable Gateway® cloning (see below), add 4 guanine (G) nucleotides to the 5′ end of the forward primer, followed by the 25 bp attB1 site, followed by 18-25 bp of template specific sequence (5′-GGGGACAAGTTTGTACAAAAAAGCAGGCT(SEQ ID NO:3)-template specific sequence-3′). For the reverse primer, add 4 guanine (G) nucleotides followed by the 25 bp attB2 site, followed by 18-25 bp of template specific sequence (5′-GGGGACCACTTTGTACAAGAAAGCTGGGT(SEQ ID NO:4)-template specific sequence-3′). Once primers are obtained for the sequence of interest, they should be diluted to about 20 μM concentration.

Also, as understood in the art, standard restriction enzyme-based cloning strategies or gene-specific primers incorporating selected restriction sites may be used to facilitate restriction enzyme-based cloning strategies to clone amplicons into an alternative entry vector (pENTR™2B, Invitrogen). Use of these primers with less non-hybridizing 5′ overhang may increase the efficiency of the initial amplification step.

### Gateway Cloning

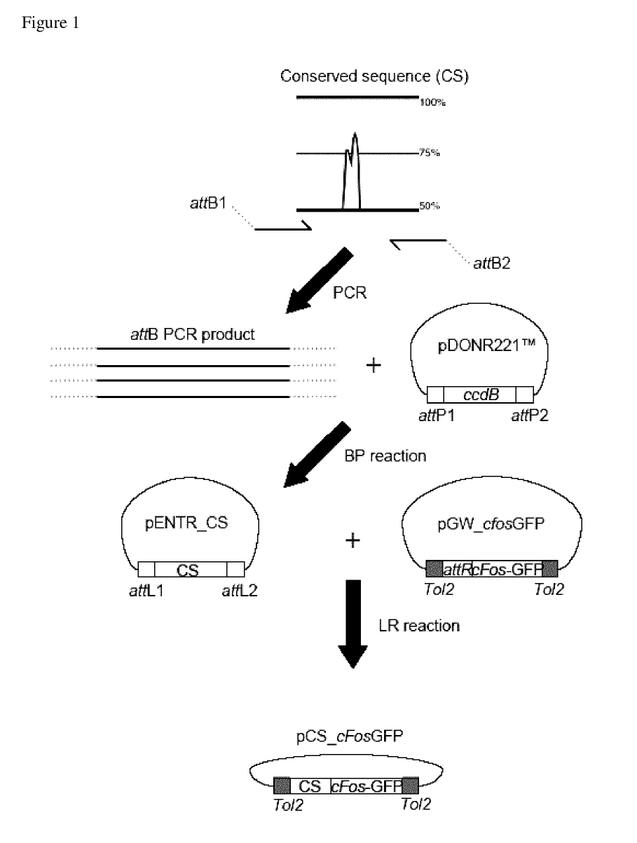

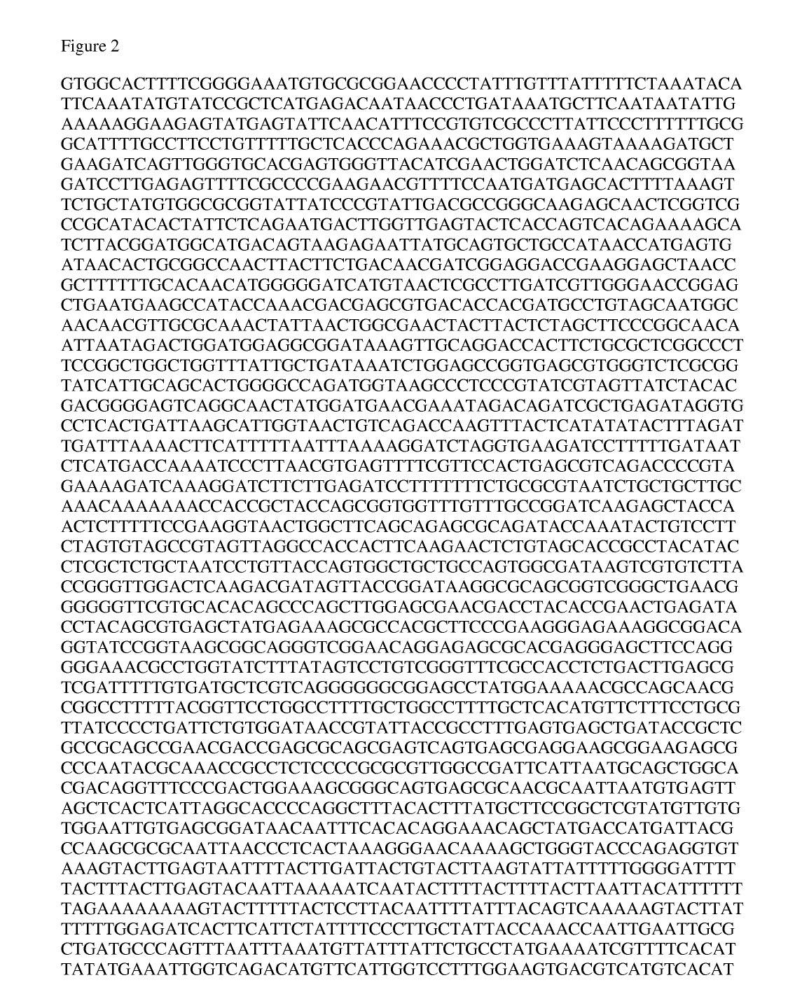

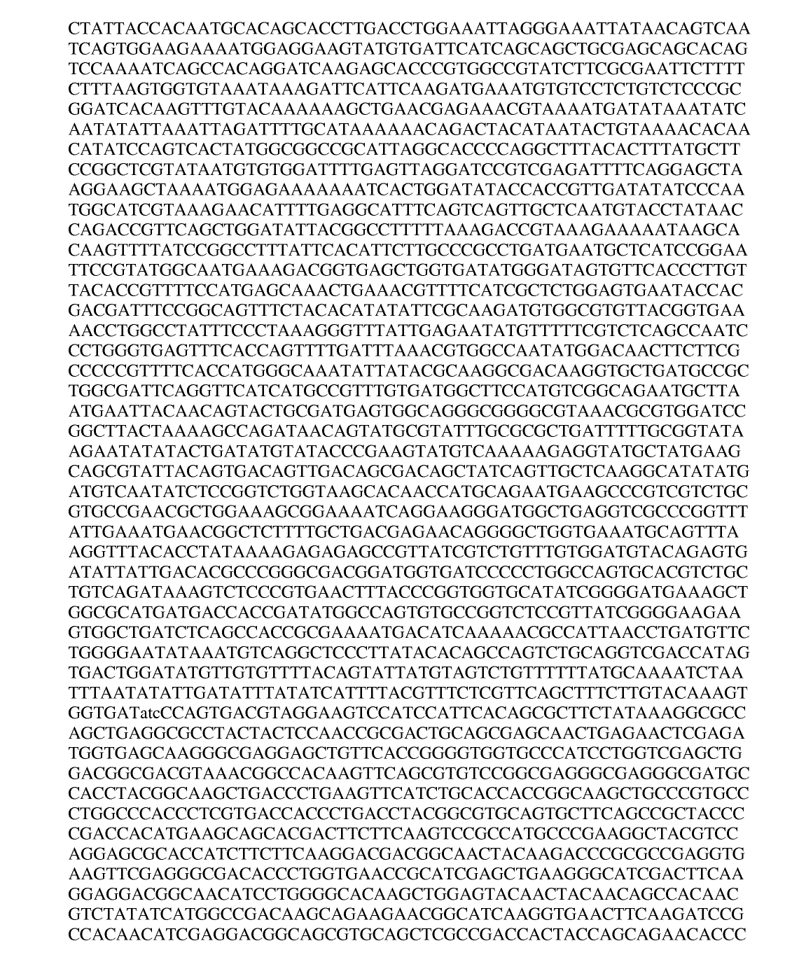

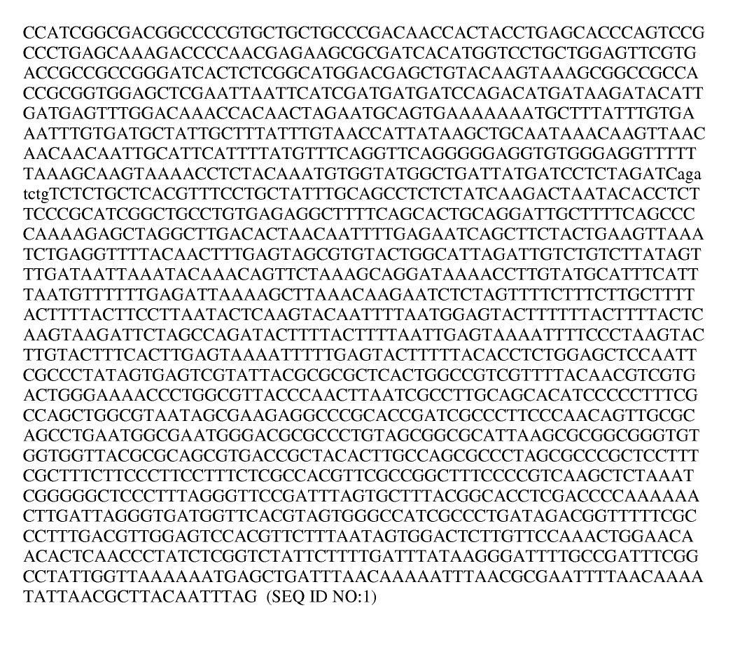

For cloning purposes, the Gateway® Technology may be used. Sequences fewer than 6 kb may be readily managed by both the Gateway® system and Tol2 transposition capabilities. Once primers are designed and the desired sequence is amplified with flanking attB sites, a recombination reaction transfers the PCR product to a donor vector pDONR™221, containing attP sites (FIG. 1). This is the BP reaction, and the resulting construct, referred to as an entry clone, contains the sequence of interest flanked by attL sites. The term “BP” is not an acronym; it refers to the recombination event that occurs between the attB and attP sites (BP) on the PCR product and the donor vector (pDONR), respectively. From the entry clone, the non-coding conserved sequence can be shuttled by LR recombination to any Gateway® ready destination vector, for example pGW_cfosEGFP, which contains a ccdB gene and chloramphenicol gene flanked by attR recombination sites (FIG. 1). As above, the term “LR” is not an acronym; it refers to the recombination event that occurs between the attL and attR sites (LR) (See FIG. 1). The ccdB gene serves as a negative selection gene for the destination vector. ccdB encodes a protein that interferes with E. coli DNA gyrase and is therefore lethal except in certain bacterial strains, such as DB3.1™ (Invitrogen). Therefore, the destination vector should only be propagated in DB3.1™ cells. When LR recombination occurs, the ccdB gene and chloramphenicol resistance gene are replaced by the sequence of interest, and therefore are able to be propagated in DH5α™ strains. Further details related to these methods are available in the manufacturer's manual on Gateway® cloning, which is available on the world wide web at the extension invitrogen.com/content.cfm?pageid=4072.

### Preparation of Injection Needles

Injection needles may be pulled from a 1.2 mm O.D. filament capillary glass, with a program designed to yield a strong tip with a fairly sharp taper, to penetrate intact chorions. The tips may be broken by hand under a stereomicroscope to an outer diameter of approximately 15 μm, using a clean razor blade and a micrometer slide to measure the diameter. Prepared needles can be made the day before injections and stored in a covered needle holding dish to keep clean.

The taper of the needles and the diameter of the tips are important factors in the ease of injections. If the needle tapers too gradually, then the tip will be too flexible to easily penetrate the chorion. Conversely, if the taper is too sharp, it will be difficult to break the tip to the correct diameter. If the tip diameters are inconsistent, then it will be necessary to recalibrate the injection volumes between needles.

Cloning Sequences of Interest into the Transposon Vector, pGW-cfosEGFP

PCR reactions may be set up as shown in the table below to amplify the non-coding conserved sequence with specific attB-containing primers described herein. Total genomic DNA or a large insert genomic clone may be used as a template.

In certain embodiments, the Takara LA Taq™ system, or similar Taq polymerase with proofreading capabilities may be used. Use of a proofreading polymerase is desirable to avoid the introduction of potentially deleterious mutations in sequences that are to be functionally evaluated, e.g., the Takara™ Taq polymerase amplifies sequences up to 20 kb in length, significantly in excess of our present requirements (0.5-2.5 kb).

An exemplary reaction mixture is shown in Table 1.

TABLE 1

| | Amount | | Final amount/ | |
|---|---|---|---|---|
| Component | (per reaction) | | concentration | |
| Sterile water | 20 | μl | | |
| 10 X LA PCR buffer | 3 | μl | 1 X | |
| dNTP mix (2.5 mM) | 4.8 | μl | 1 X | |
| attB1 forward primer (20 μM) | 0.4 | μl | 0.27 | μM |
| attB2 reverse primer (20 μM) | 0.4 | μl | 0.27 | μM |
| Genomic DNA (100 ng/μl) | 1 | μl | 100 | ng |
| Takara Taq polymerase (5 U/μl) | 0.4 | μl | 2 | units |
| TOTAL volume | 30 | μl | | |

The PCR reactions are then be transferred to a thermocycler and amplified. An exemplary PCR cycle may cycle 1 at 95° C. for 1 min; cycles 2-30 at 95° C. for 30 sec followed by 68° C. for 1 min/1 kb; and cycle 31 at 68° C. for 10 min. PCR reactions conditions can be readily modified to achieve optimal amplication results. These methods are well-understood in the art.

Following standard protocols, the entire PCR product may be run on an agarose gel and the desired amplified band excised. Further, the PCR product may be purified with the QIAquick® Gel Extraction kit (Qiagen) or equivalent, eluting the DNA from the column with about 20-50 μl of Buffer EB. This kit can be used for PCR products ranging in size from 70 bp to 10 kb. Each column is capable of binding up to 10 μg, and recovery is typically 70-80%. To determine recovery, it is useful to run 3-5 μl of the extracted DNA on an agarose gel to assess the efficiency of the extraction. The purified PCR product may then be quantified with a spectrophotometer. In general, it is desirable to use yields in excess of 25 ng/μl for subsequent cloning steps.

The Entry Vector Clone (pENTR_CS, FIG. 1) may be generated by incubating the purified PCR product containing attB recombination sites with a donor vector (pDONR™ 221) containing attP recombination sites, and the BP Clonase™ recombination enzyme, as described in the Gateway manual. The resulting construct, referred to as an Entry Clone, contains the non-coding conserved sequence of interest, flanked by attL sites (See FIG. 1). Conventional methods i.e., restriction enzyme-based cloning strategies may also be used to sub-clone PCR products or restriction fragments to create pENTR_CS.

The amplified sequence from pENTR_CS may be transferred into the pGW-cfosEGFP destination vector by LR recombination (detailed instructions of these steps are known in the art, e.g., they provided in the Gateway® manual). This vector is the universal acceptor Tol2 transposon vector, containing Gateway® attR recombination sequences, upstream of a cFos minimal promoter (Dorsky, R. et al. Dev Biol 241, 229-37 (2002)) and the EGFP coding sequence. The manufacturer also provides a positive control for the recombination-based cloning reaction. Restriction enzymes may also be used to clone sequences of appropriate size (≦6 kb) into a Gateway™ compatible entry vector (pENTR™2B), meaning that standard sequence-specific primers may be used to amplify required regions.

To verify the product of the LR recombination, approximately 500 ng of plasmid may be digested with EcoRV, using the manufacturer's recommended conditions, to release the insert. The size of the insert may be confirmed by agarose gel electrophoresis. However, as mutations introduced during amplification and cloning may influence the biological activity of the sequence being tested, sequencing is recommended to verify the sequence composition; primers used for amplification may be used for sequencing.

Once an accurate clone has been identified, plasmid DNA may be prepared using the Qiagen HiSpeed® Plasmid Midi Kit. A selected colony may be inoculated into 1 ml of LB medium (50 μg/ml Ampicillin), incubated at 37° C. with agitation (275 rpm) for 4-6 hours then 500 μl transferred to a flask containing 50 ml of LB medium (50 μg/ml Ampicillin) and further incubated at 37° C. with agitation (275 rpm) for 16 hours before extracting plasmid DNA according to manufacturer's instructions.

The plasmid may be further purified using a QIAquick® PCR Purification Kit, according to manufacturer's protocol. This additional purification may be used as embryos are often sensitive to contaminants that can be carried through standard DNA preparation protocols. Additional purification steps may be used as a means to circumvent any potential toxicity associated with injected DNAs. Equivalent kits may also be used. DNA may be eluted with 30 μL RNase-free water. RNase-free water may be purchased or prepared. Alternatively, Ultrapure™ Millipore filtered water may be used. DNA concentration may be quantified in the eluted samples by spectrophotometry, and diluted to a concentration of 125 ng/μL. The plasmid stocks may be stored for extended periods at 4° C.

RNase-free water is used to preserve the integrity of the transposase RNA at the injection stage. Early embryos are sensitive to amounts of injected plasmid DNA or impurities in plasmid preparations. The cleanliness of the plasmid DNA is critical for good survival and normal development of injected embryos, and the quantification must be accurate. Optical density ratio 260 nm:280 nm (OD_{260:280}) should be between 1.7 and 1.9. While this ratio is not an absolute indicator of DNA purity, experiments should incorporate appropriate controls (discussed later) to uncover DNA that is suspended in a solution that is toxic to the embryos.

### In Vitro Transcription of Transposase RNA

RNA encoding functional Tol2 transposase enzyme may be transcribed in vitro from the pCS-Tp vector (Kawakami, K. et al. Dev Cell 7, 133-44 (2004)). The pCS-Tp plasmid may be purified using a Qiagen Midi-Prep kit. Bacterial cultures should be established from a single colony picked from freshly streaked (≦4 weeks old) plates and prepared as described above. Approximately 10-20 μg may be linearized with NotI using manufacturer's recommended conditions. The digest may be preformed in a total volume of 100 μl, in a 1.5 ml micro-centrifuge tube.

Proteinase K may be added to the entire linearized template from above to a final concentration of 100-200 μg/ml and incubated for an additional 15 minutes at 37° C., to ensure destruction of restriction enzyme or other proteins, particularly contaminating RNases.

A phenol:chloroform extraction may be performed. An equal volume of phenol:chloroform:isoamyl alcohol (25:24:1) may be added to the sample in micro-centrifuge tube. The contents may be mixed until an emulsion forms, then centrifuged at maximum speed for 1 minute at room temperature. The aqueous (upper) phase is then transferred to a fresh micro-centrifuge tube and interface and organic phase are discarded. An equal volume of chloroform is subsequently added followed by centrifugation and recovery of the aqueous phase.

DNA is precipitated by adding sodium acetate to a final concentration of 0.3 M and 1 volume of isopropanol and incubate at −20° C. for 2-16 hours. The chilled solution may be centrifuged at maximum speed for 15 minutes at 4° C. The pellet is washed with 70% ice-cold ethanol and re-centrifuge at maximum speed for 5 minutes at 4° C. Air dry the pellet for 5 minutes in a fume hood, and re-suspend in RNase free water to yield a final concentration of 200 ng/μl-2 μg/μl.

A transcription reaction may be set up with the mMessage mMachine® Sp6 kit (Ambion) according to manufacturer's instructions. From a single reaction starting with 1 μg of template, a typical yield is 20 μg of RNA. RNA may be purified and precipitated according to kit instructions. RNA may be resuspended to a final concentration of ˜1 μg/μl, i.e. 20 μl for a single reaction, in RNase-free water, and quantified by UV spectrophotometry. Also approximately 1 μg of RNA may be analyzed by agarose gel electrophoresis to verify full-length transcription. Although a standard TAE or TBE gel is adequate for this analysis, the denaturing sample buffer included with the transcription kit should be used according to kit instructions.

The purity, integrity, and quantity of transposase RNA are critical to the success of the injections. RNA should provide an OD_{260:280} between 1.8 and 2.0. RNA may be further purified using a Qiagen RNeasy® mini kit. Separate batches of RNA may have different activities, thus it may be useful to test each new batch of RNA with a control plasmid to verify good activity. Aliquots of transposase RNA (175 ng/μl) can be stored at −80° C. (≦6 months).

### Fish Husbandry and Matings

Zebrafish injections may be performed in embryos of the strain AB (Johnson, S. & Zon, L. Methods in Cell Biology 60, 357-359 (1999)). AB zebrafish can be obtained from the Zebrafish International Resource Center (available on the world wide web at extension zfin.org).

Zebrafish may be maintained on a regular light-dark cycle, with 14 hours of light. The day prior to performing microinjections, the fish should be set up for timed matings in small breeding tanks, each consisting of a base tank, a slotted insert, and a plastic lid. Parallel rows of single sex tanks of fish can be created wherein each row should comprise tanks with either three females or two males per tank. Placement of a small plastic tree in each tank prevents males from fighting overnight. Further details regarding zebrafish husbandry and associated techniques may be obtained from in the art, for example, from The Zebrafish Book (Westerfield, M. (ed.) The Zebrafish Book (University of Oregon Press, Eugene, Oreg., 1995).

On the morning of the microinjections, shortly after the light cycle begins, 2 tanks containing 2 males and 3 females in clean system-treated water may be set up. Egg production should initiate shortly thereafter permitting the production of ≧200 eggs within 15 minutes. Timed production of good quality eggs can typically be continued over a two hour period after the normal ‘lights on’ time, by mixing tanks of males and females just prior to use. The yield of eggs depends on the light-dark cycle; females are most likely to lay shortly after the lights come on. Generally speaking, the quality and quantity of eggs laid decreases over the next several hours. Clutches of >200 eggs are preferable for injections, since they allow several experimental groups of 50 embryos to be injected, and an uninjected dish to also be set aside as a control for egg quality. Although smaller batches of eggs may be of good quality, they are less convenient for injections. Poor quality eggs will often (like unfertilized eggs) fail to progress to the 2 cell stage. These eggs should not be used for injections. However, some clutches may undergo early cell divisions and if used for injection may fail to progress through gastrulation, demonstrating the benefit of a control plate of uninjected embryos to discern whether embryo death is a consequence of injection conditions or embryo health.

To collect embryos, the slotted insert may be lifted out of the base tank and the fish placed into a new base filled with system-treated water. The embryos may be allowed to settle to the bottom of the tank. Most of the water may then be poured off and the embryos may then be poured into a Petri dish, e.g., a 60×15 mm Petri dish.

With a wide-bore, e.g., a 5¼″ glass pasteur pipet fitted with a latex bulb, the collected embryos may be sorted into Petri dishes, e.g., a 60×15 mm Petri dish, partially filled with Embryo Medium, in groups of about 50 embryos. The time of collection and the number of embryos may be marked on the lid of each dish. Generally speaking, it is convenient to inject embryos in groups of about 50 as it typically provides enough embryos expressing the construct extensively to allow characterization of the expression pattern, and a 60 mm dish has sufficient volume of water to keep about 50 embryos for 5-6 days.

The timing of injections, at the late one-cell to early two-cell stage, is important for extensive transgene expression and normal development. For ease in injecting large clutches of eggs, it is may be helpful to carefully monitor the fish and collect eggs within a few minutes of laying. Otherwise, the fish may continue to lay over an extended period, and the clutch may not be well synchronized.

Injection of Embryos with Transposons

Timing of approximately 3 hours refers to the likely productive period within which multiple clutches of eggs may be collected (as described above) plus the time taken to inject them.

Fresh injection solution may be prepared by mixing the following in a micro-centrifuge tube on ice: 1 μl transposon plasmid stock (125 ng/μl); 1 μl Transposase RNA stock (175 ng/μl); 0.5 μl Phenol red stock (2% in H₂O); and 2.5 μl RNase-free water.

Injection needles may be prepared, placed in holding dish, and filled by pipetting 500 nl drops of injection solution onto the wide end of each needle. After the liquid is drawn to the tip through capillary action, additional injection solution may be added to a total of about 1.5-2 μl. Allowing the liquid to draw to the tip before adding more liquid may help to prevent air bubbles in the needle. At least two needles may be prepared for each injection solution, depending on the number of different constructs and total number of embryos to be injected. This provides a backup in case a needle becomes blocked or breaks. In general, one needle may be used to inject approximately 100 embryos, with at least one extra needle per construct in case of breakage or blockage. The needle dish should be covered as much as possible, and a Kimwipe soaked in water may be placed in the dish to minimize evaporation of injection solution. While the maximum time that solution is stable in the needle has not been examined, no drop in efficacy was observed over a 3 hour period of injections.

A filled needle may be loaded into the hand-held needle holder of a Pneumatic Pico-Pump or similar pressure injector, configured and connected to a N₂ tank per manufacturer's instructions. Injection volumes may be calibrated by measuring the diameter of droplets expelled into mineral oil on a micrometer slide. Typically, an injection time of about 120 ms with a pressure of about 20 p.s.i. will yield a droplet of approximately 1 nl, but slight variations in needle diameter will affect these parameters and recalibration may be required between needles. Once the parameters are adjusted to give the desired injection volume, place the tip into the liquid in an injection dish and adjust the back pressure until injection solution is extruded very slowly from the tip between injections. The back pressure will prevent dilution or contamination of the injection solution in the needle.

Injections may be performed with the aid of a stereomicroscope at 6-10× magnification. In some embodiments, the embryos may be lined up an agarose injection tray to stabilize them for injection (Westerfield, M. (ed.) The Zebrafish Book (University of Oregon Press, Eugene, Oreg., 1995)). In another embodiment, a pair of fine forceps may be used to hold the embryo in place. In such circumstances, care must be taken not to put any pressure on the embryo after the needle penetrates the chorion, to avoid pushing the embryo out through the small hole. The injection needle should be pushed with steady pressure through the chorion and into the yolk of an embryo at the late one-cell or early two-cell stage. Ideally, the needle tip should be positioned in the yolk just below the blastomeres. Approximately 1 nl of injection solution should be expelled and then the needle should be withdrawn. The expelled volume should be visible as a phenol red stained drop below the blastomeres. In certain embodiments, a micromanipulator may be used to perform injections. In other embodiments, the injections may be performed by hand. Experienced personnel should be able to inject at least about 600 embryos in a 2-hour period, by collecting embryos from several successive lays. Approximately 150-200 embryos per construct may be injected. Thus 3-4 petri dishes of approximately 50 embryos per dish may be completed for each construct. Injection of larger numbers of embryos, e.g. 600 as discussed above, will likely require multiple egg collections to ensure that injected embryos are synchronized. Embryos may take up to 30 minutes to progress beyond the 2 cell stage. Embryo collection should be repeated until sufficient embryos have been collected to complete desired injections (≦200 embryos per construct) or until embryo production ceases.

After injections are completed, the embryos may be sorted by removing unfertilized eggs, damaged embryos, and failed injections (embryos with no phenol red in blastomeres). Unfertilized eggs and damaged embryos must be removed promptly to ensure normal development of the remaining embryos in the dish. Otherwise, the remaining live embryos may be killed or severely delayed in development.

### Analysis of Expression Patterns

After culture for the appropriate time, the G₀ embryos may be screened for EGFP expression. At early stages, prior to 24 hours post fertilization, the embryos can be directly observed. At later stages, when the embryos are motile and have begun hatching out of their chorions, they can be anesthetized with Tricaine (˜10 drops of 0.4% stock in 50 mm dish) to facilitate observation. Large clutches of embryos are most conveniently observed on a stereomicroscope fitted for epifluorescence, such as a Zeiss SV11 or Lumar V12. For high-resolution photography, the Lumar V12 or a compound microscope will be necessary. If fluorescent reporters are being used, it will be necessary to obtain appropriate filters to visualize the corresponding signal. One may continue observations of the live embryos throughout the first 5-6 days.

After 5-6 days, appropriate Go embryos may be selected, moved to tanks and raised to sexual maturity. The likelihood and rate of germline transmission typically correlates with extent of mosaic expression; therefore, those G₀ embryos with the most expression are selected for raising.

### Sexual Maturation of Zebrafish

Sexually mature G₀ adults may be crossed to wild type stocks to obtain germline transmission and to establish founder G1 transgenic stocks. Although this transposon-based approach results in multiple independent insertion events per G1 individual, it may be desirable to establish multiple independent G1 lines from different founders to avoid the confounding influence of position effects.

Under optimal injection conditions, the large majority (≧80%) of injected embryos will develop normally. In general, expression patterns that are consistent among at least 10-20% of embryos will be highly representative of the non-mosaic expression observed from the same constructs after germline transmission. However, detailed characterization of an expression pattern may require the establishment of transgenic lines. To insure that position effects on individual transgene insertions are not confounding the interpretation of expression patterns, multiple independent lines may be established for each construct. The term position effect refers to differences in expression that can be observed from identical transgenes because of regulatory control imposed on them by the genomic context in which they have inserted. Thus, the generation of 2 or more independent lines may be evaluated. Because of the high rate of integration of Tol2 vectors, in most cases fewer than about 20 G₀ adults need to be screened to identify more than one transgenic founder. From individual founders, germline transmission rates from <5% to >95% have been observed, although approximately 35% is more typical.

The following reagents may be employed in the methods described herein. 20× Salt Stock: The following components are added in order to 800 mL of dH₂O, allowing each salt to dissolve before adding the next one; 17.5 g NaCl, 0.75 g KCl, 2.9 g CaCl₂, 2.39 g MgSO₄, 0.41 g KH₂PO₄, 0.13 g Na₂HPO₄. dH₂O is added to a final volume of 1 L and the solution is sterile filtered and stored at 4° C.

500× Bicarbonate Stock: 1.5 g of NaHCO₃ is dissolved in 50 mL of dH₂O and stored at 4° C.

Embryo Medium (8 L): 400 mL of 20× Salt Stock is mixed with 16 mL of Bicarbonate Stock, and dH₂O to a final volume of 8 L. In some embodiments, to minimize fungal growth in embryo dishes, methylene blue (C₁₆H₁₈CIN₃S) can be added to the embryo medium. A 0.1% solution of methlyene blue may be prepared in embryo medium by adding 8 mL of Methylene Blue stock along with other stocks to an 8 L batch of Embryo Medium.

### 4. Kits

The present invention provides kits for practice of the afore-described methods. In certain embodiments, kits may comprise a vector, e.g., a Tol2 vector described herein. In some embodiments, a kit for identifying a functional noncoding interval comprises a vector comprising SEQ ID NO:1 and instructions for use. In another embodiment, a kit for identifying a functional noncoding interval comprises a vector comprising SEQ ID NO:2 and instructions for use. In some embodiments, a kit for identifying a functional noncoding interval may comprise a vector comprising SEQ ID NO:1 and a vector comprising SEQ ID NO:2 and instructions for use. Kits may additionally comprise RNA encoding the transposase. In other embodiments, a kit may comprise appropriate reagents for cloning a sequence interval into a Tol2 vector and/or introducing the vector into zebrafish. A kit may further comprise controls, buffers, and instructions for use. For example, a kit may comprise stock solutions such as a 20× salt stock, a 500× bicarbonate stock, and a embryo medium.

Kit components may be packaged for either manual or partially or wholly automated practice of the foregoing methods. In other embodiments involving kits, this invention contemplates a kit including compositions of the present invention, and optionally instructions for their use.

### Exemplification

The invention now being generally described, it will be more readily understood by reference to the following examples which are included merely for purposes of illustration of certain aspects and embodiments of the present invention, and are not intended to limit the invention in any way.

### Example 1

### Conservation of RET Regulatory Function From Human to Zebrafish in the Absence of Sequence Conservation

Evolutionary sequence conservation is an accepted criterion to identify noncoding regulatory sequences. Described herein is the use of a transposon-based transgenic assay in zebrafish to evaluate noncoding sequences at the zebrafish ret locus, conserved among teleosts, and at the human RET locus, conserved among mammals. Most teleost sequences directed ret-specific reporter gene expression, with many displaying overlapping regulatory control. The majority of human RET noncoding sequences also directed ret-specific expression in zebrafish. Thus, vast amounts of functional sequence information may exist that would not be detected by sequence similarity approaches.

A current hypothesis is that sequences conserved over greater evolutionary distances are more likely to be functional than those conserved over lesser distances (Boffelli, D. et al., Nat. Rev. Genet. 5, 456 (2004)). Many recent publications have focused attention on the regulatory potential of “ultra-conserved” noncoding sequences, conserved across great evolutionary distances, e.g., human to fugu (Woolfe, A. et al., PLoS Biol. 3, e7 (2005); Nobrega, M et al., Science 302, 413 (2003); Bagheri-Fam, S. et al., Genomics 78, 73 (2001); Baroukh, N. et al., Mamm. Genome 16, 91 (2005); Poulin, F. et al. Genomics 85, 774 (2005); de la Calle-Mustienes, E. et al., Genome Res. 15, 1061 (2005); Sandelin, A. et al., BMC Genomics 5, 99 (2004); Bejerano, G. et al., Science 304, 1321 (2004)) [≧300 million years, or average 74% protein identity (Veeramachaneni, V. and Makalowski, W. Nucleic Acids Res. 33, D442 (2005))]. These are frequently enhancers associated with developmental genes, consistent with strong selective pressure to preserve critical mechanisms. Analyses of identified sequences have generally fallen into two categories: analyses confined to mammals, with functional verification done in mice, or analyses including mammalian and teleost sequences, focusing on highly conserved sequences alignable at the extremes. However, simply because an expression pattern is preserved through evolution, it does not necessarily follow that the cis-regulatory elements controlling that expression in one species will function in a second.

Two hypotheses were tested herein. First, using selective pressure as a guide across moderate evolutionary distances, the majority of enhancers controlling expression at a particular locus can be identified by functional testing in a comprehensive, unbiased manner, and second, regulatory function of noncoding sequences will be conserved over evolutionary distances beyond the limit of overt sequence conservation.

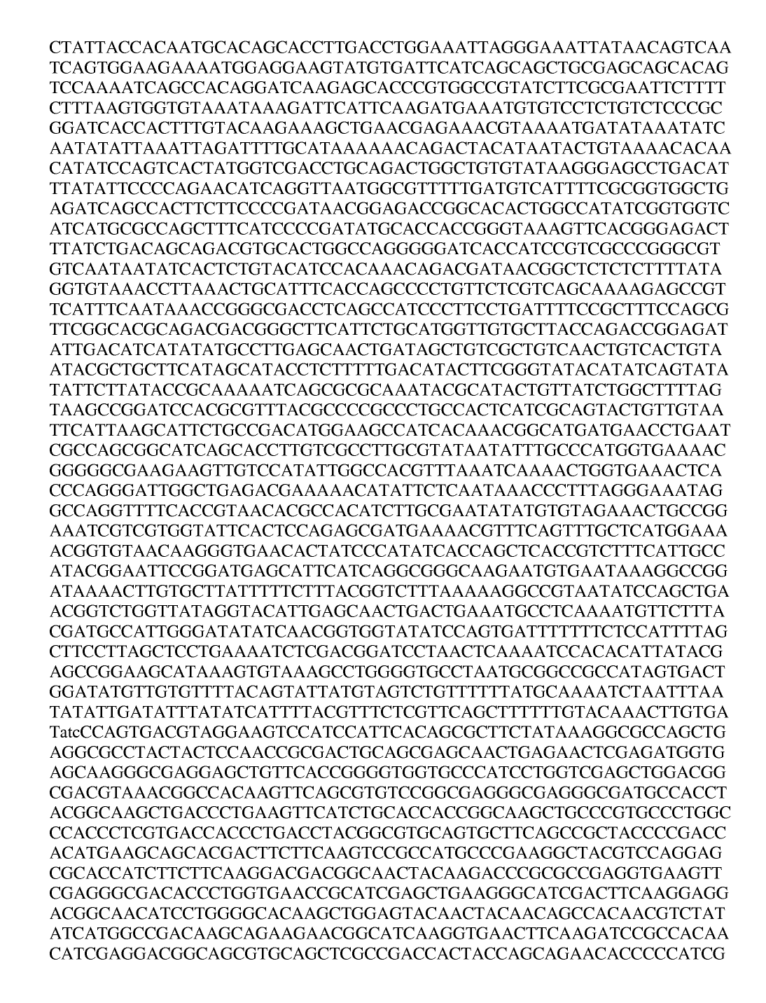

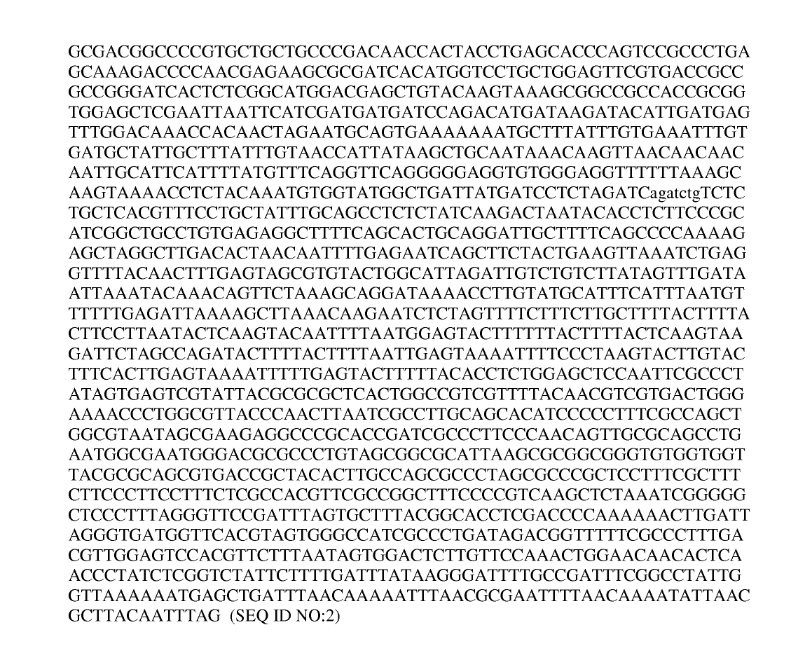

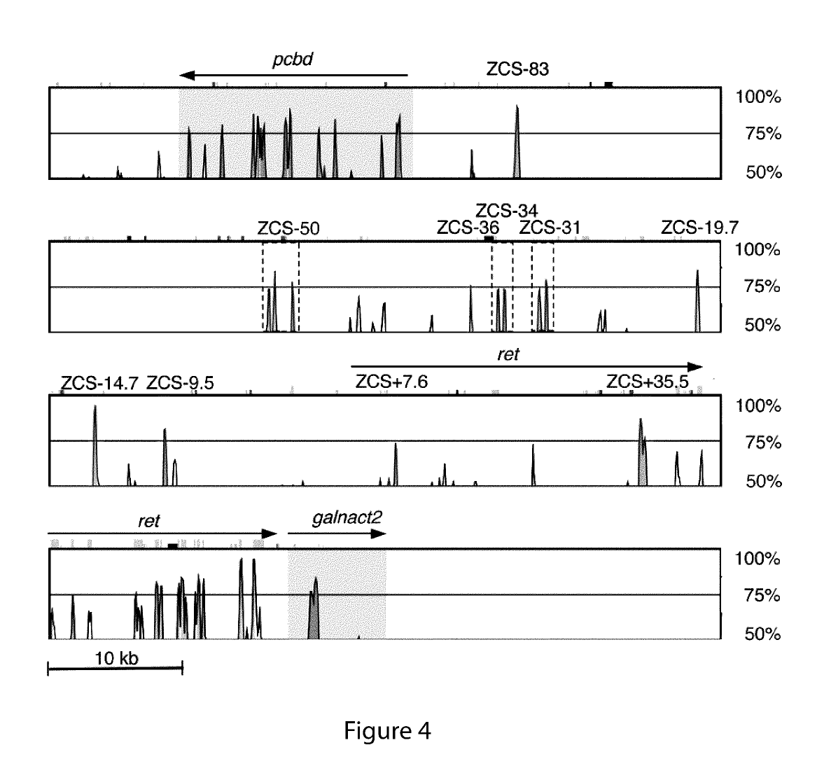

The studies described herein focused on the regulatory control of the gene encoding the RET receptor tyrosine kinase. RET is expressed in neural crest, urogenital precursors, adrenal medulla, and thyroid during embryogenesis, and in specific central and peripheral neurons and endocrine cells during development and postnatally (McCallion, A. and Chakravarti, A. in Inborn Errors of Development C. Epstein, R. Erikson, A. Wynshaw-Boris, Eds. (Oxford Univ. Press, Oxford, 2004)). Although RET expression is highly conserved across evolution (Hahn, M. and Bishop, J. Proc. Natl. Acad. Sci. U.S.A. 98, 1053 (2001); Marcos-Gutierrez, C. et al., Oncogene 14, 879 (1997); Bisgrove, B. W. et al., J. Neurobiol. 33, 749 (1997); Pachnis, V. et al., Development 119, 1005 (1993)), only the exons encoding the tyrosine kinase domain are overtly conserved [≧70%, ≧100 base pairs (bp)] from humans to zebrafish (Emison, E. et al., Nature 434, 857 (2005); McCallion, A. et al., Cold Spring Harb. Symp. Quant. Biol. 68, 373 (2003); Kashuk, C. et al., Proc. Natl. Acad. Sci. U.S.A. 102, 8949 (2005)). We first compared the genomic sequence of a ˜200-kilobase (kb) segment encompassing the zebrafish ret gene with the orthologous interval in fugu (FIG. 4), using AVID/VISTA (Frazer, K. et al., Nucleic Acids Res. 32, W273 (2004)). We generated 10 ZCS (zebrafish conserved sequence) amplicons, corresponding to 14 discrete noncoding sequences (Table 3).

These criteria were also used to identify conserved noncoding human sequences, comparing a ˜200-kb segment encompassing human RET with the orthologous genomic intervals in 12 nonhuman vertebrates (Emison, E. et al., Nature 434, 857 (2005)). Sequences shared among human and at least three nonprimate mammals were selected (Grice, E. et al., Hum. Mol. Genet. 14, 3837 (2005)). In total, 13 HCS (human conserved sequence) amplicons, encompassing 28 discrete conserved sequences (Table 4) were generated for analysis.

Although zebrafish transgenesis has been used to evaluate the regulatory potential of conserved noncoding sequences (Woolfe, A. et al., PLoS Biol. 3, e7 (2005); de la Calle-Mustienes, E. et al., Genome Res. 15, 1061 (2005); Grice, E. et al., Hum. Mol. Genet. 14, 3837 (2005)), its efficacy is compromised by mosaicism in injected (G₀) embryos. We developed a reporter vector based on the Tol2 transposon; reporter expression in G₀ embryos, driven from the ubiquitous ef1a promoter, was extensive and was dependent on transposase RNA.

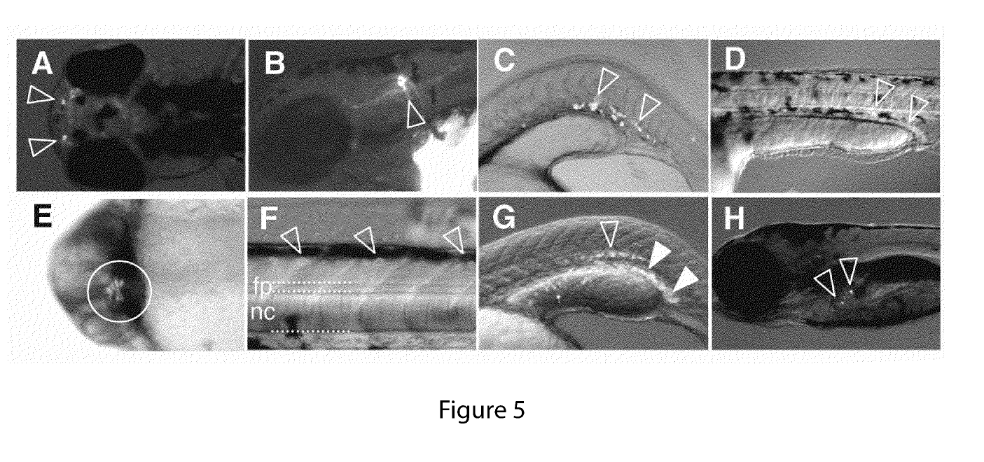

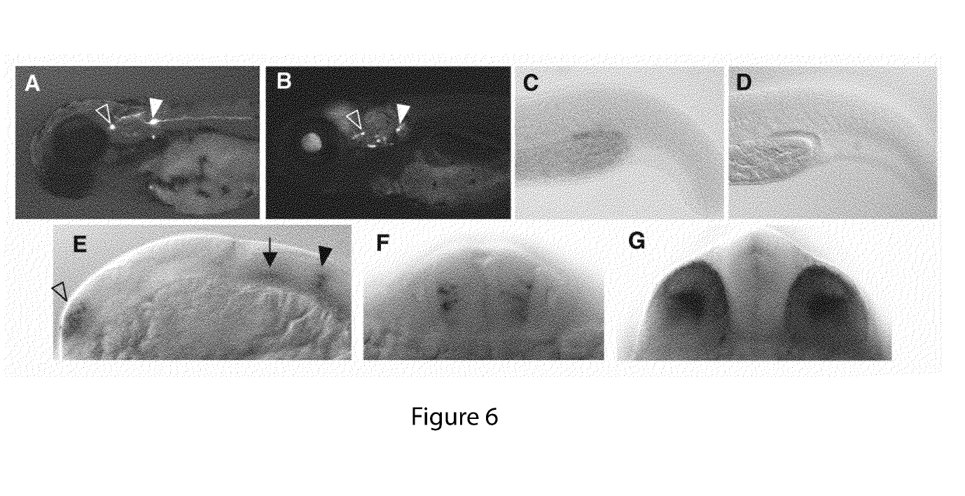

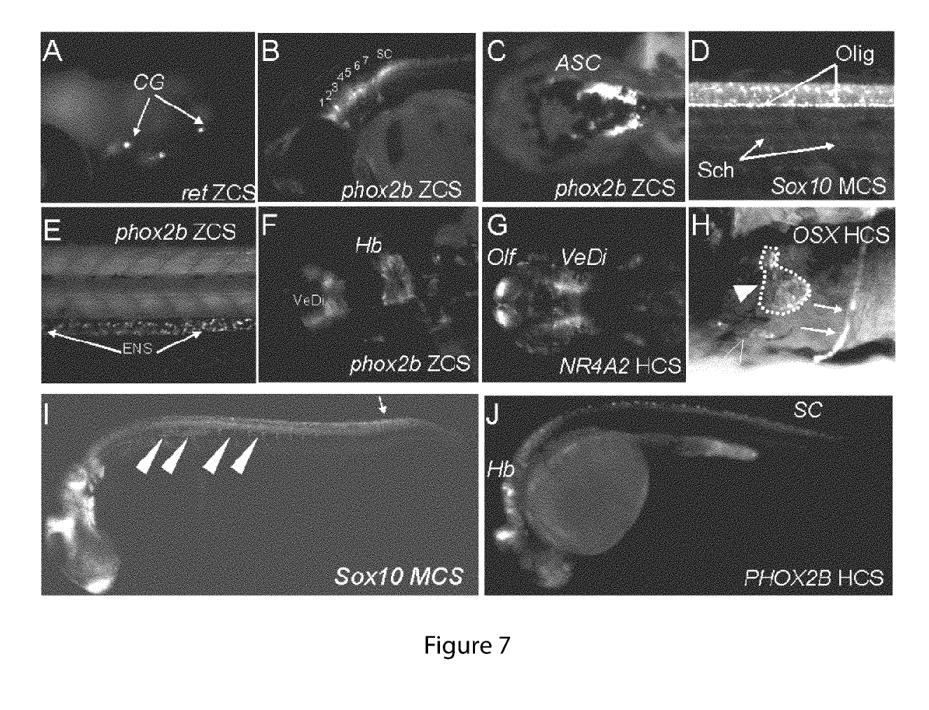

All but one ZCS amplicon drove reporter expression consistent with endogenous ret expression (Table 2). As in the mouse, zebrafish ret is expressed in sensory neurons of the cranial ganglia, motor neurons in the ventral hindbrain, cells of the hypothalamus and pituitary primordia, sensory and motor neurons in the spinal cord, and primary sensory neurons in the olfactory pit (Marcos-Gutierrez, C. et al., Oncogene 14, 879 (1997); Bisgrove, B. W. et al., J. Neurobiol. 33, 749 (1997)). Elements driving expression consistent with all of these cell populations were identified (Table 2), including small groups of cells, e.g., olfactory neurons (FIG. 5A) and lateral line placode ganglion (FIGS. 6A-B). Although ret is also expressed in amacrine and horizontal cell layers of the retina, expression in the retina of G₀ embryos was not detected with any of the tested elements.

Significant redundancy in the control of ret expression in the pronephric duct was observed (Table 2; FIGS. 5C-D). Five elements drove expression in the intermediate mesoderm or pronephric duct; one was responsible for transient early expression (FIG. 5C), one for expression in the distal duct after 3 days (FIG. 5D), and three apparently redundantly control expression in the intervening period. Although three amplicons lie within a 5-kb region upstream of ret, they function independently in this assay. Similarly all but two ZCS amplicons drove expression in one or more cell populations of the central nervous system (Table 2), wherein ret is also dynamically expressed.

Eleven out of thirteen HCS amplicons drove expression in cell populations consistent with zebrafish ret (Table 2). These included cells not present in mammals, such as the afferent neurons of the lateral line ganglia. Multiple sequences driving expression in the excretory system were also observed, despite its developmental and anatomical differences between fish and mammals (FIG. 5G). Two sequences contained within a genomic interval deleted from the rodent lineage also functioned in zebrafish, in one case driving expression in the pituitary (FIGS. 5E, 6E). Several pairs of elements drove similar expression patterns, despite lack of detectable sequence conservation (Table 2). To rule out the possibility that nonconserved sequences could fortuitously display enhancer activity, expression from vectors containing nonconserved zebrafish (n=5) or human (n=3) genomic DNA, from the RET intervals (Tables 3 and 4) was analyzed. None of these nonconserved sequences provided reproducible patterns of expression.

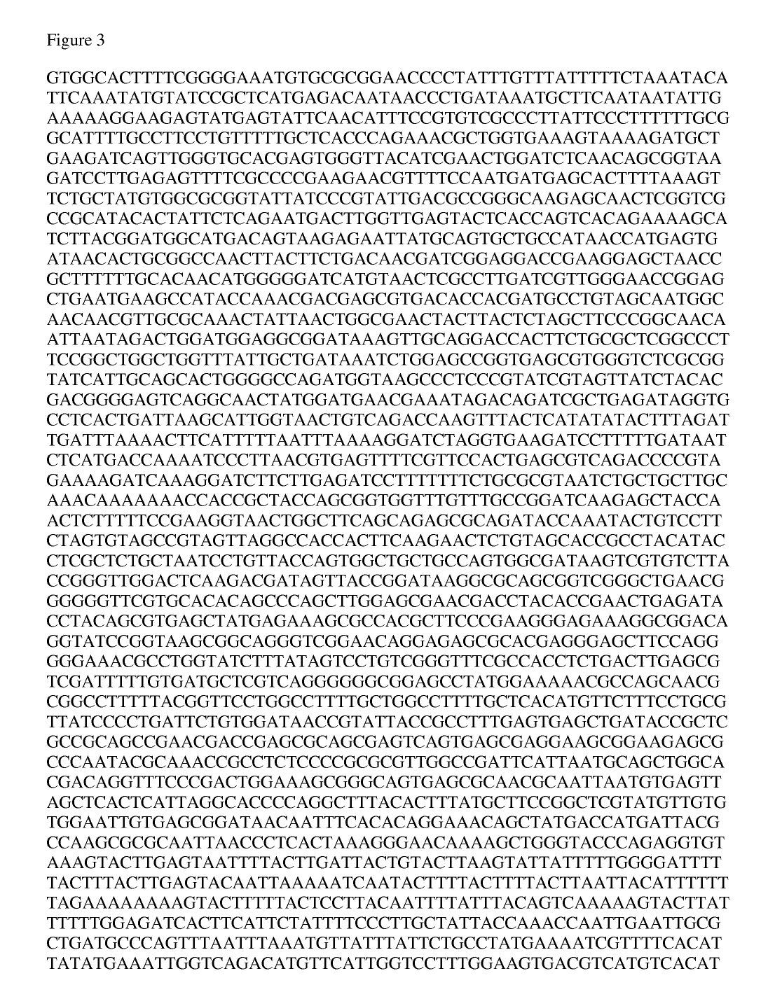

Through analysis of G₀ expression, enhancers active in small cell populations such as the cranial ganglia and olfactory neurons were identified (FIG. 5), suggesting that mosaicism is not a significant limitation. A subset of transgenes have been passed through the germline (FIGS. 6A-C and E-G), to directly compare expression in G₀ and G₁ embryos. Expression of each transgene was largely consistent with that observed in G₀ phases (FIG. 6A-B), although in some cases we observed additional expression, particularly in small groups of cells and at later time points [retina (FIG. 6G)]. In addition, many G₁ embryos were evaluated using in situ hybridization (ISH) to detect gfp transcripts, which confirmed that green fluorescent protein (GFP) signal was present in ret positive cells (FIG. 3C-D).

While still functioning as tissue-specific enhancers in zebrafish, some HCSs directed expression differing in timing or location from that of the endogenous ret gene. For example, HCS-32 drives GFP expression in dorsal spinal cord neurons, apparent between embryonic day 2 and 3. ISH analyses of G₁ transgenic embryos revealed expression at earlier stages in the posterior neural plate, where ret is not normally expressed. Additionally, two elements, HCS-23 and ZCS-50, directed expression strongly to the notochord, again not a site of endogenous ret expression. One possible reason for these discrepancies is that these elements are being assayed out of context. Also, physical proximity does not mean that these elements normally regulate ret expression. In the case of HCSs, individual transcription factor-binding sites (TFBSs) may have evolved sufficiently to display different functions (i.e., binding related proteins, binding with different affinity), reflected in altered regulatory activity of the element as a whole.

HCS function in zebrafish may arise from sequence elements ≦100 bp that are conserved but fail to meet our original criteria for identification. Consequently, sequence analysis with AVID/VISTA was repeated, reducing the window size to 30 bp. We also analyzed the RET orthologous intervals using the anchored alignment algorithms Multi-LAGAN and Shuffle-LAGAN (available on the world wide web with the extension lagan.standford.edu/lagan_wev/index), the latter designed to detect alignable sequences in the presence of inversions and rearrangements. In addition, an alignment was attempted with each RET HCS independently, in both orientations, with the zebrafish ret interval (BLAT; available on the world wide web with the extension genome.ucsc.edu/cgi-bin/hgBlat). All analyses failed to detect sequences alignable between human and zebrafish RET intervals. Further, the entire zebrafish genome was searched (available on the world wide web with the extension sanger.ac/uk/Projects/D_rerio/) for homologies to the examined HCSs. Sixty-five sequences within these HCSs of ≧20 nucleotides in length demonstrated ≧70% identity with nonorthologous, intergenic zebrafish sequences, within 100 kb of a known or predicted gene; 41 out of 65 contain conserved TFBS motifs (Table 5). However, the nonconserved HCSs were also aligned with the zebrafish genome and found alignments containing TFBSs at a similar frequency, which suggested that such analyses are not predictive of regulatory function. We posit that the responsible functional components in the conserved elements are single or multiple TFBSs (4 to 20 bp), beyond the ability of our current in silico tools to reliably detect. The data suggest that restricting in vivo functional analyses to sequences conserved over great evolutionary distances (e.g., human to teleost) detects only a small fraction of functional information in the genome.

Described herein is an efficient method to evaluate putative enhancer elements, allowing rapid assessment of in vivo function in a vertebrate embryo. This method is suitable for rapid screening of putative enhancers on a large scale, even where the orthologous zebrafish sequence is not available. Our approach represents a significant advance over previous methods because of the decreased mosaicism and improved germline transmission achieved with Tol2 vectors. The transparent external development of zebrafish facilitates dynamic analysis of reporter activity throughout embryogenesis, allowing detection of biological activity throughout development. This has allowed us to survey without bias all conserved sequences at a single, complex locus.

The data strongly suggest that functional information is conserved in vertebrate sequences at levels below the radar of large-scale genomic sequence alignment, consistent with prior anecdotal observations (Gottgens, B. et al., Nat. Biotechnol. 18, 181 (2000); Pennacchio, L. et al., Science 294, 169 (2001)). While not wishing to be bound by theory, two alternative models could be invoked to explain the data. First, overall similar expression of the RET genes could be achieved through assemblage of analogously acting, although not orthologous, enhancers. A second, more parsimonious, explanation is that orthologous enhancer elements control expression of both RET genes, but have evolved beyond recognition through small changes in TFBSs, rearrangement of sites within enhancers, or multiple coevolved changes. Examination of enhancer evolution in Drosophila species reveals examples of these types of sequence changes, confounding traditional sequence alignment approaches while preserving enhancer function across species (Berman, B. et al., Genome Biol. 5, R61 (2004); Ludwig, M. et al., Nature 403, 564 (2000); Ludwig, M. et al., PLoS Biol. 3, e93 (2005)). Comparison of human and mouse enhancer sequences suggests that similar widespread turnover of TFBSs is observed in vertebrate evolution (Pennacchio, L. et al., Science 294, 169 (2001)), although there is no corresponding functional data to confirm that such changes occur while preserving the function of the enhancers. The data cannot distinguish between these two models; however, it must be the case that largely the same set of transcription factors regulate expression of either gene, and the binding of these is conserved from mammalian to teleost enhancer elements, which allows the HCSs to function in zebrafish. These data may now significantly alter the manner in which the biological relevance of vertebrate noncoding sequences is evaluated.

### Identification of Conserved Sequences.

The RET orthologous genomic sequences described above were previously described (Emison, E. et al., Nature 434:857 (2005); Kashuk, C. et al. Proc. Natl. Acad. Sci. USA 102:8949 (2005). Conserved non-coding teleost sequences within and flanking ret were identified using VISTA (parameters ≧70%, ≧100 bp), aligning the zebrafish and fugu ret orthologous loci (˜200 kb encompassing ret). The analysis encompassed 120 kb upstream, and approximately 35 kb downstream, limited by the adjacent genes (5′, pcbd; 3′, galnact2). Results of this analysis are graphically represented in FIG. 4. All identified sequences lie within a 90 kb interval 5′ to ret and within the first ret intron. Identified sequences were PCR amplified and subcloned either independently or as small clusters when within 2 kb of one another (Boxed in green; FIG. 4). In total ten ZCS amplicons were generated for analysis.

Identification of human conserved non-coding sequences were performed in a similar manner, examining the alignment of the human RET reference sequence with 12 non-human vertebrates as described by Emison et al. (2005), selecting for analysis those sequences that were shared between human and at least 3 non-primate mammals. Sequences were name HCS* or ZCS*, where * denotes distance (kb) and relative position (+ or −; 5′ or 3′, respectively) from the transcription start site. PCR primers were designed to amplify identified sequences from the zebrafish genome (Table 3) and the human genome (Table 4). The resulting amplicons were subcloned into the transgenic construct as described in Vector Construction. HCS amplicon sequences were queried against the zebrafish genome (June 2004; DanRer2 build) using BLAT (available on the world wide web with the extension genome.ucsc.edu/cgi-bin/hgBlat). Sequence alignments between human (HCS) and zebrafish genomic sequence exceeding 70% identity were then queried for putative transcription factor binding sites using TRANSFAC via the Transcription element search system (available on the world wide web with the extension cbil.upenn.edu/tess).

### Vector Construction.

The pT2KXIGΔin plasmid was a kind gift from Koichi Kawakami (Kawakami, K. et al., Dev Cell 7:133 (2004)). To construct pT2cfosGW, the XhoI to BamHI fragment, containing the ef1a promoter and β-globin intron, was excised from pT2KXIGΔin and replaced with a minimal promoter from the mouse cFos gene (Dorsky, R. et al., Dev Biol 241:229 (2002)). The Gateway Vector Conversion kit (Invitrogen) was used to insert a cassette containing the ccdB gene and a chloramphenicol resistance gene upstream of the promoter.

Primers were designed to amplify each conserved sequence from human or zebrafish genomic DNA, and the attB1 and attB2 sequences were added to the 5′ ends of the forward and reverse primers respectively. Each PCR product was recombined first into the pDONR221 vector, and then into pT2cfosGW, using Gateway reagents (Invitrogen). The reporter vector alone showed no expression in G0 embryos.

### Embryo Injections and Analysis.

Plasmid DNAs for microinjection were purified on Geneclean® (Qbiogene) spin columns. Transposase RNA was transcribed in vitro using the mMessage mMachine® Sp6 kit (Ambion). Injection solutions were made with 25 ng/ml of transposase RNA, and 15-25 ng/ml of circular plasmid, in water. One nL of solution was injected into the yolk of wild-type embryos at the 2-cell stage. GFP expression patterns were observed in multiple embryos, generally 10-20% in each experiment. At least 200 embryos were examined for each element. Fish were cared for using standard methods (Westerfield, M. Ed., The Zebrafish Book (University of Oregon Press, Eugene, Oreg., ed. 3, 1995)). Injections were performed in AB embryos, or in a wild-type strain maintained in our facility. Germline transmission rates from G0 fish were comparable to previously published results (Kawakami, K. et al., Dev Cell 7:133 (2004)), and from some founders exceeded 95%.

### Example 2

### Identification of Enhancer Motifs Controlling Gene Expression During Skeletal Cell Differentiation

A genetic network regulating differentiation of skeletogenic cells has been delineated through mutational analysis in mice; it includes genes encoding the transcription factors Runx2, Osx, and Sox9. Direct regulatory relationships have been proposed among these transcription factors, but are mostly unsupported by any specific knowledge about the transcriptional control of these genes. Sox9 is required for chondrocyte differentiation, and may play an earlier role in formation of bipotential osteo-chondro precursors. SOX9 haploinsufficiency causes campomelic dysplasia (CD), a lethal human chondrodysplasia; deletions and translocation breakpoints associated with CD suggest that sequences as far as a megabase from SOX9 may be required for its appropriate expression. However, no specific enhancers contributing to transcriptional regulation of the human gene have been identified. The zebrafish genome contains two sox9 co-orthologs, which arose from an ancient duplication event preceding the teleost radiation.

The largely non-overlapping expression of the duplicates suggests that ancestral regulatory elements have been differentially retained during evolution of the duplicates. In particular, the elements responsible for chondrocyte expression may be associated with the jellyfish (sox9a) gene, which is required for normal chondrogenesis. This hypothesis can be tested directly through a systematic assessment of the regulatory potential of conserved non-coding elements across the Sox9 interval. Quantitative and qualitative sequence alignment algorithms have been used to analyze 500 kb of genomic sequence surrounding Sox9 from multiple vertebrates, and have identified a number of putative cis-regulatory elements. Regulatory potential was assessed for each conserved motif associated with the human gene by transgenesis in zebrafish embryos. An enhancer sufficient to direct reporter gene expression to branchial arch cartilages, which displays detectable conservation with an element associated with sox9a has been identified. Through further comparative in silico and functional analysis of sequences flanking the zebrafish sox9 genes, ancestral and novel regulatory motifs may be revealed and provide insight into the divergence of the sox9 orthologs.

### Equivalents

While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

1. A method for identifying a functional noncoding DNA sequence comprising the steps of:
(a) identifying a putative functional noncoding interval;
(b) cloning the putative functional noncoding interval into a transposon-based vector;
(c) expressing the vector in a zebrafish; and
(d) monitoring the expression of a reporter in the zebrafish,
wherein expression of the reporter indicates that the putative functional noncoding interval is a functional noncoding DNA sequence.

2. The method of claim 1, wherein the putative noncoding interval is identified by comparative sequence analysis.

3. The method of claim 2, wherein the comparative sequence analysis comprises comparing orthologous sequences to identify a conserved sequence region.

4. The method of claim 3, wherein the compared orthologous sequences are vertebrate sequences.

5. The method of claim 4, wherein the vertebrate sequences are mammalian sequences.

6. The method of claim 1, wherein the putative functional noncoding interval is identified by one or more genetic analysis.

7. The method of claim 6, wherein the one or more genetic analysis is selected from the group consisting of a transmission disequilibrium test (TDT), a linkage analysis, and an association study.

8. The method of claim 6, wherein the putative functional noncoding interval is refined by comparative sequence analysis.

9. The method of claim 8, wherein at least one orthologous sequences is compared to refine the functional noncoding interval.

10-11. (canceled)

12. The method of claim 9, wherein the interval is refined by at least an amount selected from the group consisting of 5 fold, 10 fold, and 20 fold.

13. The method of claim 6, wherein the putative functional noncoding interval identified by one or more genetic tests is not enriched by comparative sequence analysis.

14. The method of claim 1, wherein the putative functional noncoding interval is a vertebrate DNA sequence.

15. The method of claim 14, wherein the vertebrate DNA sequence is a mammalian sequence.

16. The method of claim 15, wherein the mammalian sequence is selected from the group consisting of human, non-human primate, bovine, ovine, porcine, murine, and marsupial sequence.

17. (canceled)

18. The method of claim 14, wherein the vertebrate DNA sequence is a teleost sequence.

19. The method of claim 18, wherein the teleost sequence is a zebrafish sequence.

20. The method of claim 1, wherein the putative functional noncoding interval is selected from the group consisting of cartilaginous fish, amphibian, and avian DNA sequence.

21. The method of claim 1, wherein the transposon-based vector is a Tol2 vector.

22. The method of claim 21, wherein the Tol2 vector comprises a cis-sequence for transposition, a multiple cloning site, a minimal promoter, and a reporter gene.

23-24. (canceled)

25. The method of claim 21, wherein the Tol2 vector comprises SEQ ID NO:1 or SEQ ID NO:2.

26-28. (canceled)

29. The method of claim 1, wherein the functional noncoding interval is an enhancer of gene transcription.

30. A transposon-based vector comprising SEQ ID NO:1 or SEQ ID NO:2.

31. (canceled)

32. A kit for identifying functional noncoding DNA sequences comprising a vector comprising SEQ ID NO:1 or SEQ ID NO:2 and instructions for use.

33. The kit of claim 32, further comprising an RNA encoding a transposase.

34-35. (canceled)


SEQUENCE LISTING (124 sequences)

SEQ ID NO: 1 - DNA, 6797 bp, Artificial Sequence
  Description of Artificial Sequence Synthetic construct
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtacccagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttaaatgttatttattctgcctatgaaaatcgttttcacattatatgaaattggtcagacatgttcattggtcctttggaagtgacgtcatgtcacatctattaccacaatgcacagcaccttgacctggaaattagggaaattataacagtcaatcagtggaagaaaatggaggaagtatgtgattcatcagcagctgcgagcagcacagtccaaaatcagccacaggatcaagagcacccgtggccgtatcttcgcgaattcttttctttaagtggtgtaaataaagattcattcaagatgaaatgtgtcctctgtctcccgcggatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatatcccagtgacgtaggaagtccatccattcacagcgcttctataaaggcgccagctgaggcgcctactactccaaccgcgactgcagcgagcaactgagaactcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgtctctgctcacgtttcctgctatttgcagcctctctatcaagactaatacacctcttcccgcatcggctgcctgtgagaggcttttcagcactgcaggattgcttttcagccccaaaagagctaggcttgacactaacaattttgagaatcagcttctactgaagttaaatctgaggttttacaactttgagtagcgtgtactggcattagattgtctgtcttatagtttgataattaaatacaaacagttctaaagcaggataaaaccttgtatgcatttcatttaatgttttttgagattaaaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 2 - DNA, 6797 bp, Artificial Sequence
  Description of Artificial Sequence Synthetic construct
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtacccagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttaaatgttatttattctgcctatgaaaatcgttttcacattatatgaaattggtcagacatgttcattggtcctttggaagtgacgtcatgtcacatctattaccacaatgcacagcaccttgacctggaaattagggaaattataacagtcaatcagtggaagaaaatggaggaagtatgtgattcatcagcagctgcgagcagcacagtccaaaatcagccacaggatcaagagcacccgtggccgtatcttcgcgaattcttttctttaagtggtgtaaataaagattcattcaagatgaaatgtgtcctctgtctcccgcggatcaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcgtttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtgatatcccagtgacgtaggaagtccatccattcacagcgcttctataaaggcgccagctgaggcgcctactactccaaccgcgactgcagcgagcaactgagaactcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgtctctgctcacgtttcctgctatttgcagcctctctatcaagactaatacacctcttcccgcatcggctgcctgtgagaggcttttcagcactgcaggattgcttttcagccccaaaagagctaggcttgacactaacaattttgagaatcagcttctactgaagttaaatctgaggttttacaactttgagtagcgtgtactggcattagattgtctgtcttatagtttgataattaaatacaaacagttctaaagcaggataaaaccttgtatgcatttcatttaatgttttttgagattaaaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 3 - DNA, 29 bp, Artificial Sequence
  Description of Artificial Sequence Synthetic oligonucleotide
ggggacaagtttgtacaaaaaagcaggct

SEQ ID NO: 4 - DNA, 29 bp, Artificial Sequence
  Description of Artificial Sequence Synthetic oligonucleotide
ggggaccactttgtacaagaaagctgggt

SEQ ID NO: 5 - DNA, 29 bp, Artificial Sequence
  Description of Artificial Sequence Synthetic oligonucleotide
ggggacaagtttgtacaaaaaagcaggct

SEQ ID NO: 6 - DNA, 29 bp, Artificial Sequence
  Description of Artificial Sequence Synthetic oligonucleotide
ggggaccactttgtacaagaaagctgggt

SEQ ID NO: 7 - DNA, 20 bp, Artificial Sequence
  Description of Artificial Sequence Synthetic primer
ttgatccgatgaactgcttg

SEQ ID NO: 8 - DNA, 21 bp, Artificial Sequence
  Description of Artificial Sequence Synthetic primer
tgatcattctggacagtgcaa

SEQ ID NO: 9 - DNA, 20 bp, Artificial Sequence
  Description of Artificial Sequence Synthetic primer
cccgaacactgaggtgtttt

SEQ ID NO: 10 - DNA, 20 bp, Artificial Sequence
  Description of Artificial Sequence Synthetic primer
acacaaagttctccggccta

SEQ ID NO: 11 - DNA, 21 bp, Artificial Sequence
  Description of Artificial Sequence Synthetic primer
aaacatgcctgcactgatttc

SEQ ID NO: 12 - DNA, 19 bp, Artificial Sequence
  Description of Artificial Sequence Synthetic primer
agtaacctctgccgccaat

SEQ ID NO: 13 - DNA, 22 bp, Artificial Sequence
  Description of Artificial Sequence Synthetic primer
ggaagggtcataggacactgat

SEQ ID NO: 14 - DNA, 22 bp, Artificial Sequence
  Description of Artificial Sequence Synthetic primer
gagcctttggtaacatttttca

SEQ ID NO: 15 - DNA, 22 bp, Artificial Sequence
  Description of Artificial Sequence Synthetic primer
tctgaaaagaaaatggcagtca

SEQ ID NO: 16 - DNA, 20 bp, Artificial Sequence
  Description of Artificial Sequence Synthetic primer
catgcctaagaatcccatga

SEQ ID NO: 17 - DNA, 23 bp, Artificial Sequence
  Description of Artificial Sequence Synthetic primer
gattttgcctacgcaactaagaa

SEQ ID NO: 18 - DNA, 23 bp, Artificial Sequence
  Description of Artificial Sequence Synthetic primer
gcactcttcaaacacgctttact

SEQ ID NO: 19 - DNA, 23 bp, Artificial Sequence
  Description of Artificial Sequence Synthetic primer
tccatcttggctgtaaagaagag

SEQ ID NO: 20 - DNA, 23 bp, Artificial Sequence
  Description of Artificial Sequence Synthetic primer
tcaatggaacaaagagtgtagca

SEQ ID NO: 21 - DNA, 22 bp, Artificial Sequence
  Description of Artificial Sequence Synthetic primer
cactcagtttaggtgcgacagt

SEQ ID NO: 22 - DNA, 23 bp, Artificial Sequence
  Description of Artificial Sequence Synthetic primer
ccatgtgagcaaaattatgtgaa

SEQ ID NO: 23 - DNA, 23 bp, Artificial Sequence
  Description of Artificial Sequence Synthetic primer
ttgtcgtatatgatgccagtttg

SEQ ID NO: 24 - DNA, 23 bp, Artificial Sequence
  Description of Artificial Sequence Synthetic primer
atgacatttagtcctggttttgc

SEQ ID NO: 25 - DNA, 23 bp, Artificial Sequence
  Description of Artificial Sequence Synthetic primer
ctcattaaacacatccgctttct

SEQ ID NO: 26 - DNA, 22 bp, Artificial Sequence
  Description of Artificial Sequence Synthetic primer
aggcaggaacacaggagagata

SEQ ID NO: 27 - DNA, 20 bp, Artificial Sequence
  Description of Artificial Sequence Synthetic primer
gggaaaccgattgctacaaa

SEQ ID NO: 28 - DNA, 20 bp, Artificial Sequence
  Description of Artificial Sequence Synthetic primer
gcagcattaatcttggctga

SEQ ID NO: 29 - DNA, 20 bp, Artificial Sequence
  Description of Artificial Sequence Synthetic primer
gtcacggtcacacattttgg

SEQ ID NO: 30 - DNA, 20 bp, Artificial Sequence
  Description of Artificial Sequence Synthetic primer
tccaaaagttcaagcctggt

SEQ ID NO: 31 - DNA, 20 bp, Artificial Sequence
  Description of Artificial Sequence Synthetic primer
tttttcccaaaatcgagcag

SEQ ID NO: 32 - DNA, 20 bp, Artificial Sequence
  Description of Artificial Sequence Synthetic primer
caaacttttgccgggtagtg

SEQ ID NO: 33 - DNA, 21 bp, Artificial Sequence
  Description of Artificial Sequence Synthetic primer
gggcacctaggttaccatttt

SEQ ID NO: 34 - DNA, 22 bp, Artificial Sequence
  Description of Artificial Sequence Synthetic primer
tcaggtcaaaatacccaacaga

SEQ ID NO: 35 - DNA, 20 bp, Artificial Sequence
  Description of Artificial Sequence Synthetic primer
ctcatgcggtggtgtatgtc

SEQ ID NO: 36 - DNA, 20 bp, Artificial Sequence
  Description of Artificial Sequence Synthetic primer
aacaaagggtgcatttggag

SEQ ID NO: 37 - DNA, 20 bp, Artificial Sequence
  Description of Artificial Sequence Synthetic primer
tgcagtcccatagaggaagg

SEQ ID NO: 38 - DNA, 20 bp, Artificial Sequence
  Description of Artificial Sequence Synthetic primer
gcctttctggcacacaattt

SEQ ID NO: 39 - DNA, 22 bp, Artificial Sequence
  Description of Artificial Sequence Synthetic primer
gacacaagtggagcagagacac

SEQ ID NO: 40 - DNA, 22 bp, Artificial Sequence
  Description of Artificial Sequence Synthetic primer
acaacaaaaagcccactgatct

SEQ ID NO: 41 - DNA, 22 bp, Artificial Sequence
  Description of Artificial Sequence Synthetic primer
agaggagcacacaagtgcataa

SEQ ID NO: 42 - DNA, 22 bp, Artificial Sequence
  Description of Artificial Sequence Synthetic primer
atgtggtctgtgagaagctgaa

SEQ ID NO: 43 - DNA, 22 bp, Artificial Sequence
  Description of Artificial Sequence Synthetic primer
ctagagctcaccagggtggaag

SEQ ID NO: 44 - DNA, 22 bp, Artificial Sequence
  Description of Artificial Sequence Synthetic primer
tcatgtctggaagcttctcctt

SEQ ID NO: 45 - DNA, 22 bp, Artificial Sequence
  Description of Artificial Sequence Synthetic primer
cttggtatccaggcctaaactg

SEQ ID NO: 46 - DNA, 22 bp, Artificial Sequence
  Description of Artificial Sequence Synthetic primer
tcattgttgtctgttcctggtc

SEQ ID NO: 47 - DNA, 22 bp, Artificial Sequence
  Description of Artificial Sequence Synthetic primer
gaccaggaacagacaacaatga

SEQ ID NO: 48 - DNA, 22 bp, Artificial Sequence
  Description of Artificial Sequence Synthetic primer
gaatctctctttcccccagtct

SEQ ID NO: 49 - DNA, 22 bp, Artificial Sequence
  Description of Artificial Sequence Synthetic primer
cagcttacagttccctcctgtc

SEQ ID NO: 50 - DNA, 22 bp, Artificial Sequence
  Description of Artificial Sequence Synthetic primer
tgctagagcacacacctgtctt

SEQ ID NO: 51 - DNA, 22 bp, Artificial Sequence
  Description of Artificial Sequence Synthetic primer
agagatggaacctgtctgaacc

SEQ ID NO: 52 - DNA, 22 bp, Artificial Sequence
  Description of Artificial Sequence Synthetic primer
gaggaggaggaaagaatccaat

SEQ ID NO: 53 - DNA, 24 bp, Artificial Sequence
  Description of Artificial Sequence Synthetic primer
actccttacaaagggctttctctc

SEQ ID NO: 54 - DNA, 24 bp, Artificial Sequence
  Description of Artificial Sequence Synthetic primer
cctacacagggtctccttgactac

SEQ ID NO: 55 - DNA, 20 bp, Artificial Sequence
  Description of Artificial Sequence Synthetic primer
caagcctagaggccatgaag

SEQ ID NO: 56 - DNA, 20 bp, Artificial Sequence
  Description of Artificial Sequence Synthetic primer
ccttacgatggcagagcaat

SEQ ID NO: 57 - DNA, 20 bp, Artificial Sequence
  Description of Artificial Sequence Synthetic primer
aatgggcaagaccatctcag

SEQ ID NO: 58 - DNA, 20 bp, Artificial Sequence
  Description of Artificial Sequence Synthetic primer
gtggccagtagctggaagag

SEQ ID NO: 59 - DNA, 22 bp, Artificial Sequence
  Description of Artificial Sequence Synthetic primer
attatagtcccaggcaaaagca

SEQ ID NO: 60 - DNA, 22 bp, Artificial Sequence
  Description of Artificial Sequence Synthetic primer
cctaaggatagttcctgccaga

SEQ ID NO: 61 - DNA, 22 bp, Artificial Sequence
  Description of Artificial Sequence Synthetic primer
tgtgatgaaagagggatgaatg

SEQ ID NO: 62 - DNA, 22 bp, Artificial Sequence
  Description of Artificial Sequence Synthetic primer
tccagggactagaggtaggaca

SEQ ID NO: 63 - DNA, 22 bp, Artificial Sequence
  Description of Artificial Sequence Synthetic primer
gtcttgctctgacactccacag

SEQ ID NO: 64 - DNA, 22 bp, Artificial Sequence
  Description of Artificial Sequence Synthetic primer
tggagacttcagcacctgtcta

SEQ ID NO: 65 - DNA, 22 bp, Artificial Sequence
  Description of Artificial Sequence Synthetic primer
gagcccttatgactgaatcacc

SEQ ID NO: 66 - DNA, 22 bp, Artificial Sequence
  Description of Artificial Sequence Synthetic primer
agcccctacacaggttagaaca

SEQ ID NO: 67 - DNA, 21 bp, Artificial Sequence
  Description of Artificial Sequence Synthetic primer
agcagaagcgtgtgatgactt

SEQ ID NO: 68 - DNA, 20 bp, Artificial Sequence
  Description of Artificial Sequence Synthetic primer
ctccctgtgtgtggtgtgtc

SEQ ID NO: 69 - DNA, 24 bp, Artificial Sequence
  Description of Artificial Sequence Synthetic oligonucleotide
ctaaacaaatagccaagttttcag

SEQ ID NO: 70 - DNA, 20 bp, Artificial Sequence
  Description of Artificial Sequence Synthetic oligonucleotide
cataagctctgtgcaaacat

SEQ ID NO: 71 - DNA, 34 bp, Artificial Sequence
  Description of Artificial Sequence Synthetic oligonucleotide
ttaagtgggaagttgtattttcattttggaagaa

SEQ ID NO: 72 - DNA, 34 bp, Artificial Sequence
  Description of Artificial Sequence Synthetic oligonucleotide
ggactggtgttttttcaatcaatatttttaatta

SEQ ID NO: 73 - DNA, 24 bp, Artificial Sequence
  Description of Artificial Sequence Synthetic oligonucleotide
gtcattttggaagaagaaggaggg

SEQ ID NO: 74 - DNA, 28 bp, Artificial Sequence
  Description of Artificial Sequence Synthetic oligonucleotide
ccatcaatcaatatttttaattacttac

SEQ ID NO: 75 - DNA, 20 bp, Artificial Sequence
  Description of Artificial Sequence Synthetic oligonucleotide
catttttaaaggtgaactgt

SEQ ID NO: 76 - DNA, 35 bp, Artificial Sequence
  Description of Artificial Sequence Synthetic oligonucleotide
gattgactaataaaattctccaccaactaattgat

SEQ ID NO: 77 - DNA, 25 bp, Artificial Sequence
  Description of Artificial Sequence Synthetic oligonucleotide
agaggaagaagagaaggaagaggaa

SEQ ID NO: 78 - DNA, 24 bp, Artificial Sequence
  Description of Artificial Sequence Synthetic oligonucleotide
ttttgacaagaaaacaaaccttct

SEQ ID NO: 79 - DNA, 22 bp, Artificial Sequence
  Description of Artificial Sequence Synthetic oligonucleotide
actaaagtagttttaaaaataa

SEQ ID NO: 80 - DNA, 25 bp, Artificial Sequence
  Description of Artificial Sequence Synthetic oligonucleotide
tgccagctccaggttgctggaggaa

SEQ ID NO: 81 - DNA, 20 bp, Artificial Sequence
  Description of Artificial Sequence Synthetic oligonucleotide
tactaagtagttttaaaaat

SEQ ID NO: 82 - DNA, 27 bp, Artificial Sequence
  Description of Artificial Sequence Synthetic oligonucleotide
aaagctgcttttattaagacaaaaaag

SEQ ID NO: 83 - DNA, 26 bp, Artificial Sequence
  Description of Artificial Sequence Synthetic oligonucleotide
aggttttcattaaattcctttgctcc

SEQ ID NO: 84 - DNA, 24 bp, Artificial Sequence
  Description of Artificial Sequence Synthetic oligonucleotide
gagaagaaacacctgagctggagc

SEQ ID NO: 85 - DNA, 23 bp, Artificial Sequence
  Description of Artificial Sequence Synthetic oligonucleotide
tttacaaagctgcttttatttag

SEQ ID NO: 86 - DNA, 21 bp, Artificial Sequence
  Description of Artificial Sequence Synthetic oligonucleotide
tatcagtcatttagtgggggg

SEQ ID NO: 87 - DNA, 26 bp, Artificial Sequence
  Description of Artificial Sequence Synthetic oligonucleotide
ggagaagaatgtttctgtgcttttta

SEQ ID NO: 88 - DNA, 31 bp, Artificial Sequence
  Description of Artificial Sequence Synthetic oligonucleotide
acaccaaaaatctattaaagtcataaagttc

SEQ ID NO: 89 - DNA, 27 bp, Artificial Sequence
  Description of Artificial Sequence Synthetic oligonucleotide
ttgtaaagtatccaatctacagaaaat

SEQ ID NO: 90 - DNA, 24 bp, Artificial Sequence
  Description of Artificial Sequence Synthetic oligonucleotide
ctaaagggcatctgtgtttggtct

SEQ ID NO: 91 - DNA, 30 bp, Artificial Sequence
  Description of Artificial Sequence Synthetic oligonucleotide
gttgtctaaatctgcatatctgctggacag

SEQ ID NO: 92 - DNA, 23 bp, Artificial Sequence
  Description of Artificial Sequence Synthetic oligonucleotide
cacagaagcaaaaaggaaaagaa

SEQ ID NO: 93 - DNA, 22 bp, Artificial Sequence
  Description of Artificial Sequence Synthetic oligonucleotide
tgcttgcatttacacactgtaa

SEQ ID NO: 94 - DNA, 26 bp, Artificial Sequence
  Description of Artificial Sequence Synthetic oligonucleotide
cctgtgagggacttcatatctgcagt

SEQ ID NO: 95 - DNA, 22 bp, Artificial Sequence
  Description of Artificial Sequence Synthetic oligonucleotide
agccagtctgtcttggcagctc

SEQ ID NO: 96 - DNA, 24 bp, Artificial Sequence
  Description of Artificial Sequence Synthetic oligonucleotide
gtggaggagaaaaccattaaaaat

SEQ ID NO: 97 - DNA, 23 bp, Artificial Sequence
  Description of Artificial Sequence Synthetic oligonucleotide
agtggaggagaaaaccttaaaaa

SEQ ID NO: 98 - DNA, 23 bp, Artificial Sequence
  Description of Artificial Sequence Synthetic oligonucleotide
agcagagagcaaaatggggaagt

SEQ ID NO: 99 - DNA, 22 bp, Artificial Sequence
  Description of Artificial Sequence Synthetic oligonucleotide
tcacttaaattgcctttcttct

SEQ ID NO: 100 - DNA, 20 bp, Artificial Sequence
  Description of Artificial Sequence Synthetic oligonucleotide
ctttcttctgcagaatcact

SEQ ID NO: 101 - DNA, 21 bp, Artificial Sequence
  Description of Artificial Sequence Synthetic oligonucleotide
gaggaaatcactgtttccttg

SEQ ID NO: 102 - DNA, 21 bp, Artificial Sequence
  Description of Artificial Sequence Synthetic oligonucleotide
tcacttaaattgcctttcttc

SEQ ID NO: 103 - DNA, 23 bp, Artificial Sequence
  Description of Artificial Sequence Synthetic oligonucleotide
cctctgtttgcttttgccatcaa

SEQ ID NO: 104 - DNA, 44 bp, Artificial Sequence
  Description of Artificial Sequence Synthetic oligonucleotide
ttgattcattgattcattcattggttcataaatttattcattca

SEQ ID NO: 105 - DNA, 35 bp, Artificial Sequence
  Description of Artificial Sequence Synthetic oligonucleotide
ttgattcattgattcattcattggttcttcattca

SEQ ID NO: 106 - DNA, 31 bp, Artificial Sequence
  Description of Artificial Sequence Synthetic oligonucleotide
ttgattcattgattcattcattattcattca

SEQ ID NO: 107 - DNA, 38 bp, Artificial Sequence
  Description of Artificial Sequence Synthetic oligonucleotide
ttgattcattgattcattcattggttcataaatttatt

SEQ ID NO: 108 - DNA, 28 bp, Artificial Sequence
  Description of Artificial Sequence Synthetic oligonucleotide
tgattcattgattcactgtaagagtgag

SEQ ID NO: 109 - DNA, 35 bp, Artificial Sequence
  Description of Artificial Sequence Synthetic oligonucleotide
attgattcattcattggttcataaatttattcatt

SEQ ID NO: 110 - DNA, 24 bp, Artificial Sequence
  Description of Artificial Sequence Synthetic oligonucleotide
tcgttgattcattgattcattcat

SEQ ID NO: 111 - DNA, 26 bp, Artificial Sequence
  Description of Artificial Sequence Synthetic oligonucleotide
attcattgattcattcattggttcat

SEQ ID NO: 112 - DNA, 23 bp, Artificial Sequence
  Description of Artificial Sequence Synthetic oligonucleotide
attcattgattcattcattggtt

SEQ ID NO: 113 - DNA, 25 bp, Artificial Sequence
  Description of Artificial Sequence Synthetic oligonucleotide
ggggggtgggggtggggttcacatc

SEQ ID NO: 114 - DNA, 22 bp, Artificial Sequence
  Description of Artificial Sequence Synthetic oligonucleotide
ttgattcattgattcattcatt

SEQ ID NO: 115 - DNA, 22 bp, Artificial Sequence
  Description of Artificial Sequence Synthetic oligonucleotide
ggtgggggggtgggggtggggt

SEQ ID NO: 116 - DNA, 31 bp, Artificial Sequence
  Description of Artificial Sequence Synthetic oligonucleotide
attcattcattggttcataaatttattcatt

SEQ ID NO: 117 - DNA, 24 bp, Artificial Sequence
  Description of Artificial Sequence Synthetic oligonucleotide
gctggagggaccagctggcctggg

SEQ ID NO: 118 - DNA, 22 bp, Artificial Sequence
  Description of Artificial Sequence Synthetic oligonucleotide
tcctcttactcccttccccttc

SEQ ID NO: 119 - DNA, 21 bp, Artificial Sequence
  Description of Artificial Sequence Synthetic oligonucleotide
tcgttgattcattattcattc

SEQ ID NO: 120 - DNA, 26 bp, Artificial Sequence
  Description of Artificial Sequence Synthetic oligonucleotide
tttgtaagtcagtgggagtgtctgtc

SEQ ID NO: 121 - DNA, 23 bp, Artificial Sequence
  Description of Artificial Sequence Synthetic oligonucleotide
ggaggccagtgttctgcagagtc

SEQ ID NO: 122 - DNA, 22 bp, Artificial Sequence
  Description of Artificial Sequence Synthetic oligonucleotide
gttcctcctctccttagcatgc

SEQ ID NO: 123 - DNA, 24 bp, Artificial Sequence
  Description of Artificial Sequence Synthetic oligonucleotide
gtgccaggagtgggagtgtctgtc

SEQ ID NO: 124 - DNA, 25 bp, Artificial Sequence
  Description of Artificial Sequence Synthetic oligonucleotide
gtgccaggccaccgctgtgctgcag